SEQUENCE LISTING (2 sequences)

SEQ ID NO: 1 - DNA, 1129 bp, Botryococcus sudeticus AVFF007
  misc_feature: FERM BP-11420
tacggtcgcttcggtgagtactgctatggccttcctttctacgggggacgggctcctgggattaacttctcgggatccggattccgtgatgatactttgagtaaattggagtgttcaaagcaagcctgcgctctgaacattttagcatggaatatcacgataggactctggcctatcttgttggtctgtaggaccagagtaatgattaagagggacagtcgggggcattcgtatttcattgtcagaggtgaaattcttggatttatgaaagacgaactactgcgaaagcatttgccaaggatgttttcattaatcaagaacgaaagttgggggctcgaagacgattagataccgtcgtagtctcaaccataaacgatgccgactagggattagcagacgtttcattgatgactctgctagcaccttatgtgaaaacaaagtttttgggttccggggggagtatggtcgcaaggctgaaacttaaaggaattgacggaagggcaccaccaggcgtggagcctgcggcttaatttgactcaacacgggaaaacttaccaggtccagacatagtgaggattgacagattgagagctctttcttgattctatgggtggtggtgcatggccgttcttagttggtgggttgccttgtcaggttgattccggtaacgaacgagacctcagctcctaaatagtccatgccgccttttggcggtatgctgacttcttagagggactattggcgttctagccaatggaagtatgaggcaataacaggtctgtgatgcccttagatgttctgggccgcacgcgcgctacactgatgcgttcaacgagcctatccttgaccgagaggtccgggtaatctttgaaaccgcatcgtgatggggctagactattgcaattattagtctttaacgaggaatgcctagtaagcgcgattcatcagatcgcgttgattacgtccctggtacacaccgcccgtcgctcctaccgattgggtgtgctggtgaagtgttcggatcggcaaagctgggggcaactcttgcattgccgagaagttcattaaaccctcccacctagaggaaggagaagtcgtaacaaggtttccgtaggtgaacctgcggaag

SEQ ID NO: 2 - DNA, 1650 bp, Chlorococcum sp. FFG039
  misc_feature: FERM BP-22262
ctgcttatacggtgaaactgcgaatggctcattaaatcagttatagtttatttgatggtacctcctacttggataaccgtaggaaatctagagctaatacatgcgtaaatcccgacttctggaagggacgtatttattagataaaaggccagccgggcttgcccgaccttaggcgaatcatgataacttcacgaatcgcatggccttgtgccggcgatgtttcattcaaatttctgccctatcaactttcgatggtaggatagaggcctaccatggtggtaacgggtgacggaggattagggttcgattccggagagggagcctgagaaacggctaccacatccaaggaaggcagcaggcgcgcaaattacccaatcccgacacggggaggtagtgacaataaataacaatactgggcatttatgtctggtaattggaatgagtacaatgtaaatatcttaacgagtatccattggagggcaagtctggtgccagcagccgcggtaattccagctccaatagcgtatatttaagttgttgcagttaaaaagctcgtagttggatttcgggtgcgtcgacgcggtctgcctctggtatgtactgcgctcggtgcacctttcagctgaatacgctctcctgggcttaactgcttgggaagtggagtcagcagagtgaccttgagcaaacaagagtgttcaaagcaagccgacgctctgaattttttagcatggaatcacacgataggactctggcctatcttgttggtctgtaggaccggagtaatgattaagagggacagtcgggggcattcgtatttcattgtcagaggtgaaattcttggatttatgaaagacgaacttctgcgaaagcatttgccaaggatgttttcattgatcaagaacgaaagttgggggctcgaagacgattagataccgtcgtagtctcaaccataaacgatgccgactagggattggcaggtgttccattgatgaccctgccagcaccttatgagaaatcaaagtttttgggttccggggggagtatggtcgcaaggctgaaacttaaaggaattgacggaagggcaccaccaggcgtggagcctgcggcttaatttgactcaacacggggaaacttaccaggtccagacacggggaggattgacagattgagagctctttcttgattctgtgggtggtggtgcatggccgttcttagttggtgggttgccttgtcaggttgattccggtaacgaacgagacctcagcctgctaaatagtcacgggtaccttggtacacgcctgacttcttagagggactattggcgtctagtcaatggaagtgtgaggcaataacaggtctgtgatgcccttagatgttctgggccgcacgcgcgctacactgatgcattcaacgagcctatccttggccgagaggtccgggtaatctttgaaactgcatcgtgatggggatagattattgcaattattagtcttcaacgaggaatgcctagtaagcgcaagtcatcagcttgcgttgattacgtccctgccctttgtacacaccgcccgtcgctcctaccgattgggtgtgctggtgaagtgttcggattggctccagggatgggcaaccactcctgttgctgagaagaac

(19) United States
(12) Patent Application Publication
(10) Pub. No.: US 2016/0194600 A1
(43) Pub. Date: Jul. 7, 2016

(54) METHOD FOR LIQUID-SURFACE FLOATING CULTURE OF MICROALGAE USING MICROALGAE ON BOTTOM SURFACE AS SEED ALGAE, METHOD FOR PRODUCING ALGAL BIOMASS, AND MICROALGA

(71) Applicant: FUJIFILM Corporation, Tokyo (JP)

(72) Inventors: Hideyuki KANEHARA, Ashigarakami-gun (JP); Tadashi MATSUNAGA, Tokyo (JP); Tsuyoshi TANAKA, Tokyo (JP); Masayoshi TANAKA, Tokyo (JP); Masaki MUTO, Tokyo (JP)

(73) Assignee: FUJIFILM Corporation, Tokyo (JP)

(21) Appl. No.: 15/074,966

(22) Filed: Mar. 18, 2016

Related U.S. Application Data
(63) Continuation of application No. PCT/JP2014/074957, filed on Sep. 19, 2014.

(30) Foreign Application Priority Data
Sep. 20, 2013 (JP) 2013-194973
Mar. 20, 2014 (JP) 2014-058126

(51) Int. Cl. C12N 1/12; C12P 7/64

(52) U.S. Cl. CPC C12N 1/12; C12P 7/64

(57) ABSTRACT

An object of the present invention is to provide a method for performing fluid surface-floating culture of microalgae without supplying new seed algae. Microalgae on the bottom surface are used as seed algae. That is, there is provided a method for culturing microalgae which includes: a first culture process of culturing microalgae in a medium within a culture vessel, forming a biofilm on the liquid surface of the medium, and maintaining the microalgae on the bottom surface of the culture vessel; a process of collecting at least a part of the biofilm on the liquid surface formed in the first culture process and leaving at least some microalgae on the bottom surface inside the culture vessel; and a second culture process of culturing the microalgae remaining on the bottom surface within the identical culture vessel, and forming a biofilm on the liquid surface of the medium. In addition, the present invention provides novel microalgae which can form a biofilm on the liquid surface.

### CROSS-REFERENCE TO RELATED APPLICATIONS

This application is a Continuation of PCT International Application No. PCT/JP2014/074957 filed on Sep. 19, 2014, which claims priority under 35 U.S.C §119(a) to Japanese Patent Application No. 2013-194973 filed on Sep. 20, 2013 and Japanese Patent Application No. 2014-058126 filed on Mar. 20, 2014. Each of the above application(s) is hereby expressly incorporated by reference, in its entirety, into the present application.

### BACKGROUND OF THE INVENTION

1. Field of the Invention

The present invention relates to liquid surface-floating culture of microalgae. More specifically, in the present invention, after collecting algal bodies as products, it is possible to start next culturing without performing supply of new seed algae. The present invention is useful in a field of producing algal biomass or the like.

2. Description of the Related Art

In the related art, culturing of microalgae is performed while the microalgae are dispersed in a medium (hereinafter, referred to as dispersion culture). However, agitation energy for dispersion, or a centrifugal separator, a flocculant, or the like for collecting microalgae which have been dispersed is required. For this reason, costs of culturing and collecting microalgae increase significantly. Furthermore, in a case of extracting a product (useful substance) from the collected microalgae, the moisture content in the collected substance increases. As a result, problems, in that the amount of energy to be input into a drying process in order to increase the concentration of microalgae is extremely high, have been pointed out, and therefore, costs increase and a sufficient energy balance is not reached energy balance (Mitsufumi MATSUMOTO, of al., The 62th Annual Meeting of the Society for Biotechnology, Japan (2010) Topics Collection, p. 33 and Yoshimi OJIMA, “Microalgae Change World”, [online], Jan. 14, 2011, TS UKUBA SCIENCE, Internet <http://tsukubascience.com/seibutsu/sourui_ga_sekai_wo_kaeru/>). For this reason, the process of extracting various products from microalgae has not been commercialized, except for some expensive products. In addition, in a case where the products are inexpensive products such as fuel (oil), there is no example of successful commercialization from the viewpoint of input energy, costs, and energy balance.

Examples of a method for improving the above-described problems include a method for efficiently supplying seed algae. However, in the conventional method, it is necessary to prepare a culture vessel for seed algae. Continuation of culturing is generally performed by collecting a part of microalgal dispersion liquid within the culture vessel and adding a fresh medium with substantially the same amount as that of the collected microalgal dispersion liquid to the culture vessel. That is, culturing is performed without newly adding seed algae (JP2013-90598A and JP2005-40035A).

Regarding the genus Chlorococcum (Chlorococcum sp.), there is a disclosure of accumulation of fatty acids in Journal of Algal Biomass Utilization (2012), Vol. 3, pp. 12, and there is a disclosure that it is possible to grow the genus Chlorococcum, even at a high concentration of carbon dioxide in Oceanological Studies, 1998, Vol. 27, No. 1, Publisher: Index Copernicus, p. 17. However, there is no disclosure of the genus Chlorococcum floating on the liquid surface. In addition, RK261 of the genus Chlorococcum is disclosed in Journal of Applied Microbiology (2010), Vol. 108, pp. 1421-1429, but there is no disclosure of the RK261 of the genus Chlorococcum floating on the liquid surface.

### SUMMARY OF THE INVENTION

The present inventors have suggested a liquid surface-floating culture method which is related to a method for culturing microalgae and is used for forming a biofilm on the liquid surface and culturing while making the biofilm floating on the liquid surface. In the liquid surface-floating culture method, it is possible to collect algal bodies without using a device such as a centrifugal separator since a biofilm of microalgae floats on the liquid surface, and no energy for stirring is required since the liquid surface-floating culture method is stationary culture, and therefore, the liquid surface-floating culture method is a culture method performed with low energy at low costs compared to the conventional method.

The present inventors showed that, in the liquid surface-floating culture method, it is possible to perform continuous culturing without newly supplying seed algae through a method for collecting some water surface algae on the liquid surface and starting culturing again in a state in which a part of the collected substance is made to float on the liquid surface, or through a method for performing collection while leaving some water surface algae. An object to be solved by the present invention is to provide a new method for starting culturing without newly preparing and supplying seed algae.

It is considered that a large amount of microalgal biofilm on the bottom surface of a culture vessel has a certain thickness and is adhered to the bottom surface. For this reason, microalgae within the biofilm exist in a high density, and therefore, it is considered that there are many areas which are rarely involved in proliferation. Another object of the present invention is to provide a method for solving this problem.

In a medium which has been used for culturing at least once or more times, there is a case where a nutrient source for culturing is insufficient, or there is a possibility that a substance inhibiting culturing may be included. Still another object of the present invention is to provide a method for solving this problem.

Furthermore, when performing culturing using bottom surface algae as seed algae, a case, in which the quantity of microalgae on the liquid surface is decreased in accordance with increase of the number of times of culturing (number of times of repeating culturing), has been found. Still another object of the present invention is to provide a method for solving this problem.

In addition, still another object of the present invention is to provide microalgae with a high proliferation rate and high oil content.

The present inventors have completed the present invention by finding a method for repeatedly performing the liquid surface-floating culture method using bottom surface algae without newly preparing seed algae. The present invention provides the following.

[1] A method for culturing microalgae, including:

a first culture step of culturing microalgae in a medium within a culture vessel, forming a biofilm on the liquid surface of the medium, and maintaining the microalgae on the bottom surface of the culture vessel;

a step of collecting at least a part of the biofilm on the liquid surface formed in the first culture step and leaving at least some microalgae on the bottom surface inside the culture vessel; and

a second culture step of culturing the microalgae remaining on the bottom surface within the identical culture vessel, and forming a biofilm on the liquid surface of the medium.

[2] The culture method according to [1], further including:

a step of removing at least a part of the medium used in the first culture step and adding a fresh medium.

[3] The culture method according to [2], in which the concentration of at least one component contained in the fresh medium is at least 50 times the concentration of the medium used in the first culture step.
[4] The culture method according to [3],

in which the at least one component which is contained in the fresh medium and of which the concentration is at least 50 times the concentration of the medium used in the first culture step is a compound including any one selected from the group consisting of nitrogen, phosphorus, potassium, calcium, magnesium, sulfur, and iron.

[5] The culture method according to any one of [2] to [4],

in which the removal of the at least a part of the medium used in the first culture step is removal of at least a part of a medium existing in a region between the liquid surface and the bottom surface.

[6] The culture method according to any one of [1] to [5],

in which the microalgae on the bottom surface include adhesive microalgae, and

in which the culture method further includes a step of separating at least some adhesive microalgae from the top of the bottom surface.

[7] The culture method according to any one of [1] to [6],

in which the second culture step includes a step of collecting at least a part of the biofilm formed on the liquid surface and leaving at least some microalgae on the bottom surface inside the culture vessel, and

in which the culture method further includes third and subsequent repeating culture steps of culturing the remaining microalgae on the bottom surface within the identical culture vessel and repeating formation of a biofilm on the liquid surface of the medium.

[8] The culture method according to [7],

in which, in second and subsequent culture steps, the quantity of microalgae existing on the bottom surface as seed algae is less than or equal to 5 mg/cm².

[9] The culture method according to [8],

in which, in the second and subsequent culture steps, at least a part of a medium used in an immediately prior culture step is removed, and at this time, the quantity of microalgae existing on the bottom surface as seed algae is set to less than or equal to 5 mg/cm² by removing both non-adhesive microalgae on the bottom surface and the at least the part of the medium thereof.

[10] The culture method according to [4] or [5],

in which the microalgae on the bottom surface include adhesive microalgae and non-adhesive microalgae, and

in which, when the at least a part of the medium used in the first culture step are removed, both the non-adhesive microalgae on the bottom surface and the at least a part of the medium thereof are removed.

[11] The culture method according to any one of [1] to [10],

in which the quantity of microalgae existing on the bottom surface as seed algae is greater than or equal to 0.1 mg/cm².

[12] The culture method according to any one of [1] to [11],

in which the quantity of microalgae existing on the bottom surface as seed algae is greater than or equal to 2 mg/cm².

[13] The culture method according to any one of [1] to [12],

in which the microalgae are microorganisms containing oil.

[14] The culture method according to any one of [1] to [13],

in which the microalgae are green algae.

[15] The culture method according to any one of [1] to [14],

in which the microalgae belong to Botryococcus sp., Chlamydomonas sp., Chlorococcum sp., Chlamydomonad sp., Tetracystis sp., Characium sp., or Protosiphon sp.

[16] The culture method according to any one of [1] to [15],

in which the microalgae belong to Botryococcus sudeticus or Chlorococcum sp.

[17] The culture method according to any one of [1] to [16],

in which the microalgae are Botryococcus sudeticus FERM BP-11420 or microalgae having taxonomically the same properties as those of Botryococcus sudeticus FERM BP-11420, or are Chlorococcum sp. FERM BP-22262 or microalgae having taxonomically the same properties as those of Chlorococcum sp. FERM BP-22262.

[18] Microalgae of which the identity with base sequences of a partial region corresponding to Chlorococcum sp. RK261 among base sequences encoding a gene region of 18S rRNA is 95.00% to 99.99% or which belong to Chlorococcum sp.,

in which the 18S rRNA gene thereof has sequence identity of at least 99.94% with polynucleotide formed of a base sequence of SEQ ID No: 2.

[19] Microalgae which are Chlorococcum sp. FFG039 strains (accession number of FERM BP-22262) or microalgae having taxonomically the same properties as those of Chlorococcum sp. FFG039 strains.
[20] A method for producing algal biomass, including:

a culture step including the culture method according to any one of [1] to [17]; and

a step of collecting a biofilm on the liquid surface formed in the second culture step.

[21] The production method according to [20],

in which the algal biomass is oil.

By using bottom surface algae as seed algae, it is possible to repeatedly perform liquid surface-floating culture without newly preparing seed algae. Furthermore, a microalgal biofilm is subdivided by dispersing microalgae on the bottom surface of a culture vessel in a medium. In addition, the opportunity of contact of microalgae with the medium is increased through separation of microalgae from contact with the surface of the culture vessel. Therefore, it is possible to improve the proliferation rate of microalgae. Furthermore, nutrient components required for culturing are supplied to the medium using a characteristic medium replacement method in the liquid surface-floating culture method. In addition, the concentration of a substance inhibiting culturing which is considered to be contained in the medium decreases. As a result, the culturing can be favorably performed. In addition, with removal of non-adhesive bottom surface algae or excess microalgae, it is possible to avoid a problem relating to a decrease in the amount of microalgal biomass on the liquid surface which is generated in accordance with increase of the number of times of culturing. In addition, it is possible to provide microalgae with high proliferation rate and high oil content.

### BRIEF DESCRIPTION OF THE DRAWINGS

FIGS. 1A to 1H are schematic views of the present invention. FIG. 1A shows a state in which a suspension liquid of microalgae is put into a culture vessel; FIG. 1B shows a state in which the microalgae are deposited to the bottom surface of the culture vessel after the lapse of time of several seconds to several tens of minutes while allowing the microalgae to stand; FIG. 1C shows a state in which microalgae on the liquid surface and the bottom surface have proliferated by performing culturing; FIG. 1D shows a state in which a first substrate is brought into contact with the microalgae on the liquid surface, that is, collection is started through transfer; FIG. 1E shows a state in which the transferred microalgal biofilm is taken out of the culture vessel; FIG. 1F shows a state in which the microalgal biofilm on the liquid surface is collected using a second substrate; FIG. 1G shows a state in which the substrate containing the collected substance of algal bodies is taken out of the culture vessel after collecting the microalgal biofilm within the culture vessel using the second substrate; and FIG. 1H shows a state in which the microalgal biofilm on the liquid surface is removed.

FIG. 2 is a composition of a CSiFF04 medium.

FIG. 3 shows, in a liquid surface-floating culture method, the quantity of dry alga bodies after culturing in a case where the culturing has been started using bottom surface algae as seed algae, and in a case where the culturing has been started using input algal bodies which have been prepared by performing suspension treatment. The culturing has been performed four times. In the former case, culturing has been started using the bottom surface algae as seed algae without using new microalgae, and in the latter case, culturing has been started using a new culture vessel for each culturing and using input algal bodies which have been prepared by performing suspension treatment at 5×10⁵ cells/mL.

FIG. 4 is a view in which data, in the case of using the bottom surface algae as seed algae, from the data plotted in FIG. 3, is plotted again by the quantity of dry alga bodies with respect to the number of days of culturing.

FIG. 5 is a view in which the quantity of dry alga bodies with respect to the number of days of culturing in a case where liquid surface-floating culture is performed is plotted for every number of days of culturing with respect to water surface algae and the bottom surface algae.

FIG. 6 is a view in which second primary culture is started using different amounts of bottom surface algae, which have been obtained from FIG. 5, as seed algae, and the quantity of algal bodies on the liquid surface is plotted with respect to these amounts of bottom surface algae.

FIG. 7 is a part of a base sequence (SEQ ID No: 1) of a gene which encodes 18S rRNA of Botryococcus sudeticus AVFF007 strains of microalgae.

FIG. 8 shows a state of liquid surface-floating culture of FFG039 strains.

FIGS. 9 A and 9B show microscopic photographs of Chlorococcum sp. FFG039 strains

FIG. 9A shows a general state and FIG. 9B shows a state in which zoospores are released and proliferated.

FIG. 10 is a gene sequence of the FFG039 strains obtained through 18S rDNA analysis.

FIG. 11 is a genealogical tree of the FFG039 strains.

### DESCRIPTION OF THE PREFERRED EMBODIMENTS

Hereinafter, a preferred embodiment of a culture method for microalgae will be described in detail. The numerical range represented by “˜” means a range including numerical values denoted before and after “˜” as a lower limit value and an upper limit value.

[Method of Present Invention]

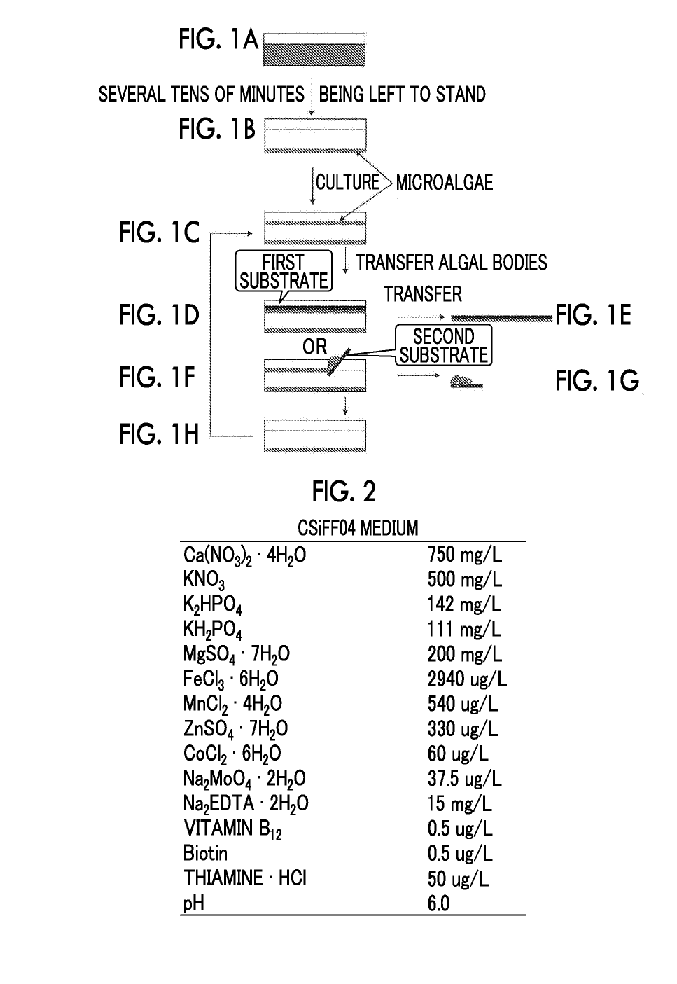

A basic culture method of the present invention is shown in FIGS. 1A to 1H. This schematic view is provided in order to describe the present invention, and therefore, some sections of this drawing are denoted by being simplified.

As shown in FIG. 1A, a suspension liquid of microalgae is prepared and put into a culture vessel. Next, when the culture vessel is in a stationary state, as shown in FIG. 1B, the microalgae sink to the bottom surface of the culture vessel within several seconds to several tens of minutes depending on the types of microalgae. The sinking of microalgae to the bottom surface means that the majority of microalgae sink to the bottom surface and does not mean a state in which microalgae completely disappear from the top of the liquid surface, the middle of the solution, the side surface of the culture vessel, or other surfaces. In this state, when the microalgae are subjected to stationary culture in this state for a while, a biofilm formed of the microalgae is formed on the liquid surface as shown in FIG. 1C (first culture process). The film-like structure changes to a three-dimensional structure in accordance with the progress of the culturing depending on the culture conditions. The change is continuous. In addition, as shown in FIG. 1C, the microalgae also exist on the bottom surface of the culture vessel. Moreover, although it is not shown in the drawing, the microalgae also exist on the side surface of the culture vessel or other surfaces. Moreover, although the number of microalgae existing is small, the microalgae also exist in the medium.

Next, the microalgal biomass on the liquid surface is collected. There is a method for collecting the microalgal biomass through a transferring method using a first substrate as shown in FIG. 1D, and a method for collecting through deposition method using a second substrate as shown in FIG. 1F. The states in which the substrates are taken out of the culture vessel are respectively states of FIGS. 1E and 1G. In the schematic view, the substrates to which microalgae adhere are moved outside the culture vessel. However, the collected substance may be collected from the substrates within the culture vessel.

The state after the biofilm on the liquid surface is collected is FIG. 1H. Here, microalgae remain on the bottom surface of the culture vessel. When the culturing is performed for a while using these microalgae, a biofilm formed of the microalgae is formed on the liquid surface as shown in FIG. 1C (second culture process). In this manner, culturing is repeated plural times, and culturing while avoiding various problems occurring during the repeated culturing is a characteristic of the present invention.

[Repetitive Culturing of Microalgal Biofilm]

The state after the microalgal biofilm on the liquid surface of FIG. 1D or 1F is collected becomes the state of FIG. 1H. Here, culturing can be performed again using microalgae, which remain on the bottom surface as seed algae, many times as long as nutrient components for proliferation remain in the medium. However, there is a high possibility that the proliferation rate becomes slow if the concentration of microalgae is too low. Therefore, in such a case, it is possible to newly add a medium thereto, to replace at least a part of a medium with another medium, and to add solid nutrient components or highly concentrated nutrient components to a medium.

[Microalgae]

Microalgae of the present invention indicate minute algae of which the individual existence cannot be identified with the naked eye. As the classification of microalgae, there is no particular limitation as long as the microalgae have an ability of forming a biofilm on the liquid surface, and either prokaryote or eukaryote may be used.

The microalgae are not particularly limited, and any microalgae can be appropriately selected in accordance with the purpose. Examples thereof include the division Cyanophyta, the division Glaucophyta, the division Rhodophyta, the division Chlorophyta, the division Cryptophyta, the division Haptophyta, the division Heterokontophyta, the division Dinophyta, the division Euglenophyta, and the division Chlorarachniophyta. These may be used alone or in a combination of two or more thereof. Among these, as the above-described microalgae, the division Chlorophyta is preferable, and green algae are more preferable.

As the microalgae, the genus Haematococcus (Haematococcus sp.), the genus Chlamydomonas (Chlamydomonas sp.), the genus Chlorococcum (Chlorococcum sp.), and the genus Botryococcus (Botryococcus sp.) are more preferable in terms of production of biomass.

The above-described method of obtaining microalgae is not particularly limited, and any method can be appropriately selected in accordance with the purpose. Examples thereof include a method of collecting microalgae in nature, a method of using a commercially available product, and a method of obtaining microalgae from a culture collection or a depositary institution. Microalgae used in the present invention are preferably microalgae obtained through a purification process. The purification process is a process which is performed for the purpose of making microalgae be a single type, and it is unnecessary to make microalgae be completely a single type.

In the present invention, among the above-described microalgae, microalgae which can produce useful substances are preferable. Particularly, microalgae which produce an intermediate or a final product for a pharmaceutical product, a cosmetic, or a health food product; a raw material used in synthetic chemistry; a hydrocarbon compound or triglyceride; an oily substance such as fatty acid compound; gas such as hydrogen; and the like are preferable. In the present invention, in some cases, these are called products. Furthermore, in the present invention, it is preferable to use microalgae which satisfy either of good culturing on the liquid surface and good recovery from the liquid surface; possession of a high proliferation rate; a high oil content ratio; little odor at least during culturing; and no generation of poisonous substances being confirmed.

[Biofilm]

The biofilm in the present invention refers to a structure of microalgae (microalgae aggregation or microalgae film) which is adhered to the surface of rock or the like. Besides these, in the present invention, a film-like structure or a three-dimensional structure, which is formed of microalgae existing on the surface such as the liquid surface having fluidity, is also called a biofilm. A biofilm in nature also contains debris or pieces of plants besides target microalgae. The biofilm of the present invention may contain these as long as the biofilm is a sample which has been obtained through a purification process. However, ideally, it is more preferable that the biofilm is formed of only the microalgae according to the present invention and a substance such as an intracellular matrix which is secreted during the proliferation of the microalgae. In addition, microalgae on the bottom surface can also be called a biofilm as long as the microalgae form a film-like structure.

In addition, it is preferable that the biofilm is configured such that individual microalgae are adhered to each other directly or via a substance (for example, polysaccharides) such as the intercellular matrix. In many cases, such a film-like structure is generally denoted as a biofilm or the like.

In the present invention, it is necessary to use microalgae capable of forming a biofilm on the liquid surface. Preferred examples of such microalgae include Botryococcus sudeticus or the genus Chlorococcum. More Specific examples thereof include Botryococcus sudeticus AVFF007 strains (hereinafter, simply referred to as AVFF007 strains) or FFG039 strains. As a result of analyzing a gene sequence which encodes 18S rRNA, the FFG039 strains are Chlorococcum sp.

[AVFF007 Strains]

The AVFF007 strains as microalgae used in Examples of the present specification are internationally deposited to Patent Organism Depository of the National Institute of Advanced Industrial Science and Technology (Central 6, 1-1, Higashi 1-chome, Tsukuba-shi, Ibaraki-ken, Japan) dated Sep. 28, 2011 with a accession number of FERM BP-11420 under the Budapest Treaty, by FUJIFILM Corporation (26-30, Nishiazabu 2-chome, Minato-ku, Tokyo, Japan). The work of the Patent Organism Depository of the National Institute of Advanced Industrial Science and Technology has been handed over to the Patent Organism Depositary of the National Institute of Technology and Evaluation (Room No. 120, 2-5-8 Kazusakamatari, Kisarazu-shi, Chiba-ken, Japan) from Apr. 1, 2012.

The AVFF007 strains are novel strains of freshwater microalgae which have been isolated from a freshwater pond in Kyoto, Japan by the present inventors. A part (SEQ ID No: 1, FIG. 7) of a base sequence of the 18S rRNA gene was analyzed using BLAST based on data of the National Center for Biotechnology Information (NCBI). As a result, the part of the base sequence thereof was identified as microalgae which were closely related to Botryococcus sp. UTEX2629 (Botryococcus sudeticus) strains (homologous to an 1109 base on the AVFF007 strain side among an 1118 base on the UTEX2629 strain side). The AVFF007 strains are microalgae which are also closely related to Characiopodium sp. Mary 9/21 T-3w, and there is also a possibility that the name of the AVFF007 strains may be changed to the genus Characiopodium. In this case, the name of the AVFF007 strains is regarded to be changed thereto. In addition, in a case where the name of the AVFF007 strains is changed to a name other than the genus Characiopodium, the name of the AVFF007 strains in the present invention is also regarded as being changed thereto.

In the present invention, it is possible to use strains having taxonomically the same properties as those of the AVFF007 strains. The taxonomic properties of the AVFF007 strains are shown below.

Taxonomic Properties of AVFF007 Strains

### 1. Morphological Properties

The AVFF007 strains have a green circular shape. The AVFF007 strains have floating properties, and therefore, can proliferate on the liquid surface and the bottom surface. The AVFF007 strains proliferate on the liquid surface and form a film-like structure. The size of an AVFF007 strain is 4 μm to 30 μm (the size of algal bodies on the liquid surface is comparatively large and the size of algal bodies on the bottom surface is comparatively small). Bubbles are formed on the liquid surface in accordance with the proliferation and overlap each other to form a three-dimensional structure. Oil is produced.

### 2. Culturing Properties (Culture Method)

(1) Medium: CSiFF04 (which is obtained by improving a CSi medium; and of which the composition is shown in FIG. 2 and the pH is adjusted to 6.0 using NaOH or HCl. The medium can be sterilized at 121° C. for 10 minutes).
(2) Culture temperature: a favorable temperature is 23° C. and culturing can be performed at less than or equal to 37° C.
(3) The culture period (the period until the culturing generally reaches a stationary phase) depends on the quantity of algal bodies which has been initially used, and is 2 weeks to 1 month. In general, culturing can be performed at 1×10⁵ cells/mL
(4) Culture method: stationary culture is suitable.
(5) Light-requiring properties: Necessary. Intensity of light: 4000 lux to 15000 lux. Bright and dark cycle: 12 hours for time for a bright period and 12 hours for time for a dark period. During subculture, it is possible to perform the culturing at 4000 lux.

The AVFF007 strains can be stored through the subculture in accordance with the above-described culturing properties (culture method). The subculture can be performed by collecting microalgae which float on the liquid surface and performing dispersion such as pipetting, and then, dispersing the microalgae in a fresh medium. Immediately after the subculture, the microalgae are sunk on the bottom surface of a culture vessel, and start to form a biofilm on the liquid surface after about one week. Proliferation can be performed by making the microalgae floating on the liquid surface immediately after the subculture. The cycle for the subculture is about one month. Subculture is performed when the microalgae exhibit yellow color.

As strains having taxonomically the same properties as those of the AVFF007 strains, microalgae are included of which the 18S rRNA gene has sequence identity of at least 95.0%, preferably 98.0%, more preferably 99.0%, still more preferably 99.5%, and most preferably 99.9% with polynucleotide formed of a base sequence of SEQ ID No: 1.

The sequence identity mentioned regarding the base sequence in the present invention means a percentage of the number of common bases which are coincident with each other between two arrayed bases within a region in a case where the two bases are arrayed in an optimal mode. That is, the identity can be calculated through an equation of identity=(number of coincident bases/total number of bases)×100 and can be calculated using an algorithm which is commercially available or open to the public. Search and analysis with respect to the identity of the base sequence can be performed using a well-known algorithm or program for those skilled in the art. In a case of using a program, those skilled in the art can appropriately set the parameter. Alternately, a default parameter of each program may be used. Specific techniques for the analysis method thereof are also well known to those skilled in the art.

[FFG039 Strains]

The FFG039 strains as microalgae used in Examples of the present specification are collected by the present inventors from Nara Prefecture in Japan. The FFG039 strains have good proliferating properties and are excellent in oil productivity compared to the AVFF007 strains. In addition, the FFG039 strains have characteristics in that the structure of the biofilm is hardly destroyed and it is easy to collect the FFG039 strains. The FFG039 strains are Chlorococcum sp. As a result of analysis of the gene sequence of 18S rRNA, the FFG039 strains are species closely related to RK261 strains (Chlorococcum sp. RK261) of the genus Chlorococcum as microalgae. In the present invention, newly isolated microalgae are named Chlorococcum sp. FFG039. It is more preferable that the identity with base sequences of a partial region corresponding to RK261 of the genus Chlorococcum among base sequences encoding a gene region of the microalgae according to the present invention is 95.00% to 99.99%. The “partial region” referred to herein means a region having greater than or equal to 1000 base sequences. When testing the identity, use of every base sequence results in the highest reliability for the test of the identity. However, determining every base sequence is technically and financially difficult except for an extremely small number of species of organisms. In addition, only a specific portion (specifically, the vicinity of base sequences corresponding to base sequences of the Chlorococcum sp. FFG039 strains (hereinafter, also simply referred to as FFG039 strains) which are set as a comparison target to be described below) of the base sequences of the RK261 strains of the genus Chlorococcum has not been disclosed. Furthermore, in general, it is considered that there can be attribution if about 1000 base sequences are read. From the above, the identity has been tested through the comparison with the base sequences of a “partial region” in the present invention, and it is considered that the reliability thereof is sufficiently high. The Japanese name of Chlorococcum is based on the Japanese name disclosed in Freshwater algae, written by Takaaki YAMAGISHI, UCHIDA ROKAKUHO PUBLISHING CO., LTD.

The FFG039 strains as microalgae used in Examples of the present specification are internationally deposited to the Patent Organism Depositary of the National Institute of Technology and Evaluation (Room No. 120, 2-5-8 Kazusakamatari, Kisarazu-shi, Chiba-ken, Japan) dated Feb. 6, 2014 with a accession number of FERM BP-22262 under the Budapest Treaty, by FUJIFILM Corporation (26-30, Nishiazabu 2-chome, Minato-ku, Tokyo, Japan).

The FFG039 strains are novel strains of freshwater microalgae which have been isolated from a pond in Kyoto in Japan by the present inventors and belong to the genus Chlorococcum.

Hereinafter, the method of isolating the microalgae (hereinafter, also referred to as purification) and circumstances in which it has been determined that the FFG039 strains of the microalgae are novel strains will be described.

[Purification of FFG039 Strains of Microalgae]

Natural fresh water was collected from a pond in Nara Prefecture by putting it into a 5 mL tube for homogenizing (TM-655S, Tomy Seiko Co., Ltd.). 100 μL of the collected natural fresh water was added to a 24 hole plate (microorganism culture plate 1-8355-02, As One Corporation) into which 1.9 mL of a medium, in which a CSiFF04 medium shown in FIG. 2 was put. The plate was placed on a plant bioshelf for tissue culture (AV152261-12-2, Ikeda Scientific Co., Ltd.) and was cultured at 23° C. under continuous irradiation with 4000 lux light. After approximately one month, a green aggregation was generated in wells of the 24 hole plate. The aggregation was observed using an optical microscope and it was confirmed that there are a large number of microorganisms.

1 g of agarose (Invitrogen, UltraPure™ Agarose) was weighed out, and 200 mL of a CSiFF04 medium was put into a 500 mL conical flask. The CSiFF04 medium was subjected to an autoclave treatment for 10 minutes at 121° C., and approximately 20 mL of the CSiFF04 medium at a time was added to an Azunol Petri dish (1-8549-04, As One Corporation) in a clean bench before being cooled and hardened to produce agarose gel.

The solution containing the microalgae in the 24 hole plate was diluted, and the solution was made to adhere to a loop portion of a disposable stick (1-4633-12, As One Corporation) and was applied to the prepared agarose gel to prepare a Petri dish in which the microalgae were applied to the agarose gel.

The Petri dish was placed on a plant bioshelf for tissue culture and was cultured at 23° C. under continuous irradiation with 4000 lux light. After approximately 2 weeks, a green colony appeared on the agarose gel. The colony was adhered to a distal end of a sterilized bamboo skewer (1-5980-01, As One Corporation), and then, was suspended in the wells of the 24 hole plate into each of which 2 mL of the CSiFF04 medium was put. The 24 hole plate containing microalgae prepared in this manner was placed on a plant bioshelf for tissue culture and was cultured at 23° C. under continuous irradiation with 4000 lux light. After approximately 2 weeks, the aqueous solution in the wells exhibited a green color. A small amount of solution was collected from all of the wells, the microalgae were observed using an optical microscope, and it was concluded that purification had been performed in wells, in which it was considered that there was only a single microalga.

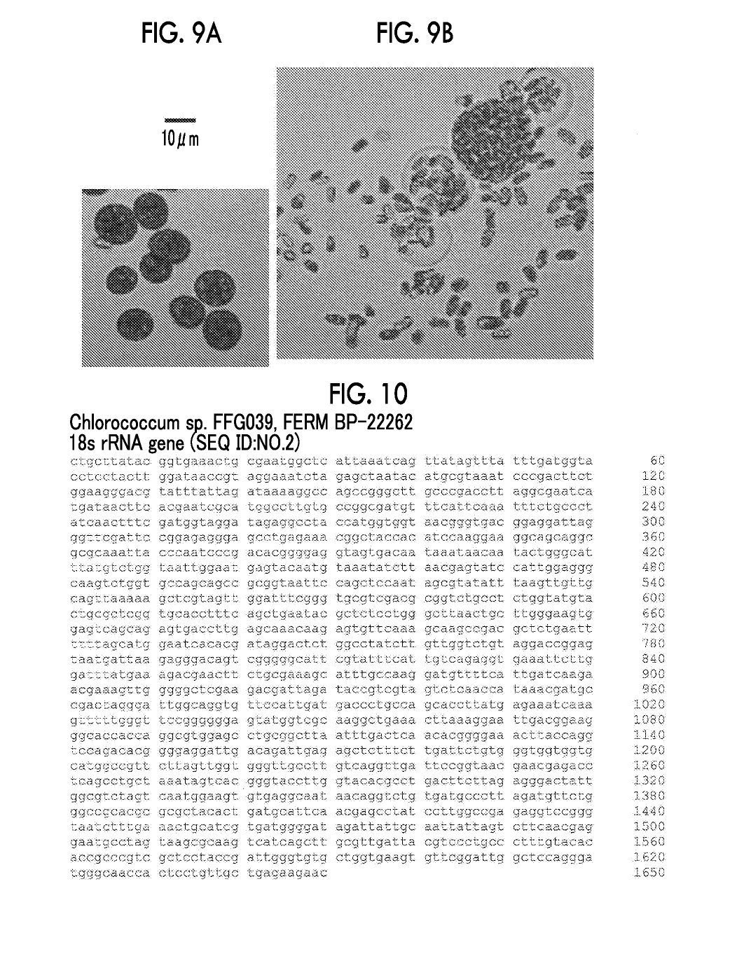

In addition, a microphotograph of FFG039 strains at magnification of 40 is shown in FIGS. 9A and 9B. FIG. 9A shows an ordinary state and FIG. 9B shows that the FFG039 strains proliferate by releasing a large number of zoospores.

[Morphological Properties]

All microalgae sink to the bottom surface if the microalgae are left for a while after performing a dispersion treatment.

If the microalgae are cultured for a while, microalgae floating on the liquid surface appear. Accordingly, microalgae are divided into microalgae having sunk to the bottom surface and microalgae floating on the liquid surface. If further culturing is performed continuously, a film-like structure appears on the liquid surface. If the culturing is performed further, a three-dimensional structure appears.

All the microalgae on the liquid surface and on the bottom surface have a spherical shape and have different size distributions.

The microalgae have cohesiveness and form a large colony.

The microalgae are green and the color thereof turns to yellow in accordance with the progress of the culturing.

There is little odor in collected substances and during the culturing.

[Culturing Properties]

During the cell proliferation, the cells proliferate through zoospores. A large number of zoospores are generated from one cell.

It is possible to perform photoautotrophic culture through photosynthesis.

Nitrogen, phosphorus, potassium, calcium, magnesium, sulfur, manganese, and iron are essential for proliferation. In addition, inclusion of zinc, cobalt, molybdenum, and boron makes the proliferation favorable. Addition of vitamins also promotes the proliferation.

[Physiological Properties]

Oil accumulates in an algal body at a maximum close to 40 wt % in terms of dry weight proportion.

In the oil, hydrocarbon compounds and fatty acids accumulate. The fatty acids produce palmitic acid, palmitoleic acid, oleic acid, vaccenic acid, linoleic acid, linolenic acid, and the like, and particularly, palmitic acid and oleic acid are main components. The hydrocarbon compounds produce decane, heptadecane, and the like.

FFG039 strains dyed with Nile Red were observed by a fluorescence microscope. Then, it was confirmed that there was oil which was colored with the Nile Red as a bright fluorescent light-emitting region in algal bodies in a fluorescent visual field. The oil can accumulate in a comparatively wider region within an alga body cell.

Identification of the FFG039 strains was further performed through the following method.

(Identification of FFG039 Strains of Micro Algae)

The culture method of the FFG039 strains was as follows. 50 mL of a CSiFF04 medium was introduced into a conical flask with a 100 mL capacity, 0.5 mL of an FFG039 strain solution at a concentration of 1000×10⁴ cells/mL was added thereto, and shaking culture was performed under light irradiation for 14 days at 25° C.

In order to obtain a dry powder of the FFG039 strains, centrifugal operation was performed on 40 mL of the medium containing the FFG039 strains obtained as described above using a centrifuge (MX-300 (Tomy Seiko Co., Ltd.) for 10 minutes at a centrifugal force of 6000×g below 4° C. After removing a supernatant, the solid body was frozen together with the container using liquid nitrogen. Then, the total quantity of frozen solid body was transferred to a mortar which was chilled in advance using liquid nitrogen, and was ground using a pestle which was chilled in advance using liquid nitrogen.

The extraction of DNA from the microalgae was performed using DNeasy Plant Mini Kit (manufactured by Qiagen) according to the described manual. The purity and the amount of the extracted DNA were measured using e-spect (manufactured by Malcom Co., Ltd.). It was confirmed that the extracted DNA achieved the index of a purification degree which is A₂₆₀ₙₘ/A₂₈₀ₙₘ=1.8 or greater, and about 5 ng/μL of DNA was taken.

There was no problem in the purification degree of the extracted DNA, and thus, a sample for PCR was prepared by diluting the DNA 10⁴ times in ultrapure water. An 18S rRNA gene region (rDNA region) was used as the sample for PCR. A cycle including 10 seconds at 98° C., 50 seconds at 60° C., and 10 seconds at 72° C. was performed 30 times for the PCR using a GeneAmp PCR System 9700 (manufactured by Applied Biosystems). An enzyme used herein was Prime Star Max (manufactured by Takara Bio Inc.). It was confirmed through 1% agarose electrophoresis that the obtained PCR product was a single band.

The purification of the PCR product was performed using a PCR purification kit (manufactured by Qiagen). The method was carried out through the method described in the manual. In order to check the purification degree and whether the PCR reaction was sufficiently performed, the purity and the amount of the DNA were measured using e-spect. It was determined that there was no problem since the measured purification degree was A₂₆₀ₙₘ/A₂₈₀ₙₘ=1.8 or greater.

Next, the purified substance was used as a template and a cycle sequence was performed using a BigDye Terminator v3.1 Cycle Sequencing kit (manufactured by Applied Biosystems). The manual was referred to for the condition. The base sequence of the obtained reactant was decoded using ABI PRISM 3100-Avant Genetic Analyzer (manufactured by Applied Biosystems).

Identity analysis was performed using basic local alignment search tool (BLAST). The method thereof was as follows. BLAST searching for the above-described sequence was conducted on the whole base sequence information in the data of the National Center for Biotechnology Information (NCBI). A species of an organism having the highest identity was regarded as a closely related species of the FFG039 strains. Only the base sequence (1650 base, SEQ ID No: 1) which was set as a comparison target is shown in FIG. 10. Specifically, several bases at both the ends of the decoded base sequence were not set as a comparison target for the BLAST analysis, and thus are not shown in FIG. 10. The upper left of the base sequence shown in FIG. 10 is a 5′-terminal and the lower right thereof is a 3′-terminal.

As a result of the identity analysis, the above-described sequence had the identity (that is, 99.94% identity) to a 1649 base on the FFG039 strain side among a Chlorococcum sp. RK261 strain side and 1650 bases on the Chlorococcum sp. RK261 strain side. Accordingly, the FFG039 strains were classified as microalgae closely related to the Chlorococcum sp. RK261 strains.

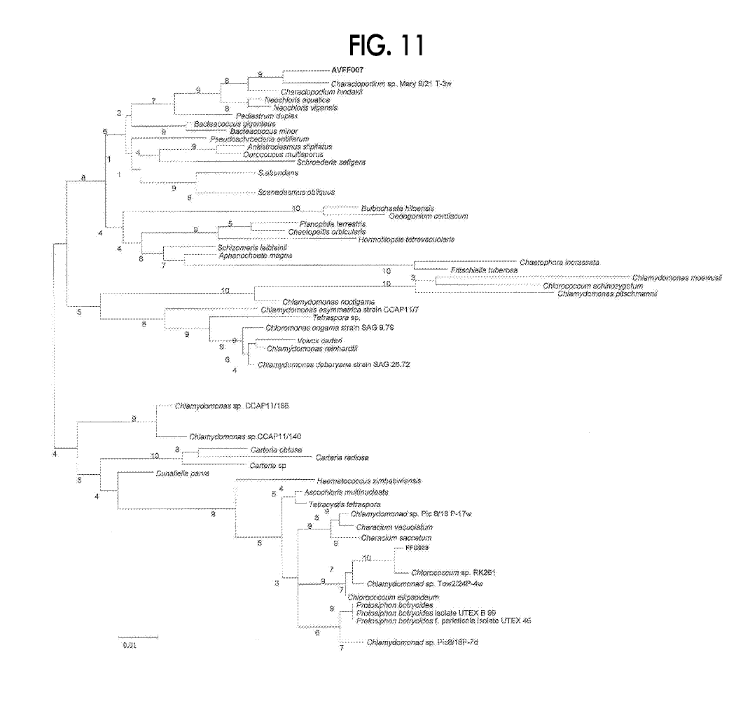

The systemic diagram obtained from the results of the above-described analysis is shown in FIG. 11. In a case where the name of the Chlorococcum is changed, similarly, it is regarded that the name of the FFG039 strains is also changed in the present invention.

In the present invention, it is possible to use strains having taxonomically the same properties as those of the FFG039 strains. The taxonomic properties of the FFG039 strains are shown below.

Taxonomic Properties of FFG039 Strains

### 1. Morphological Properties

The AVFF007 strains have a circular shape. When stationary culture is performed, a film-like structure is formed on the liquid surface. Oil is produced.

### 2. Culturing Properties (Culture Method)

(1) Medium: a CSiFF04 medium or a CSi improved medium (150 mg/L of Ca(NO₃)₂.4H₂O, 100 mg/L of KNO₃, 28.4 mg/L of K₂HPO₄, 22.2 mg/L of KH₂PO₄, 40 mg/L of MgSO₄.7H₂O, 588 ug/L of FeCl₃.6H₂O, 108 ug/L of MnCl₂.4H₂O, 66 ug/L of ZnSO₄.7H₂O, 12 ug/L of CoCl₂.6H₂O, 7.5 ug/L of Na₂MoO₄.2H₂O, 3 mg/L of Na₂EDTA.2H₂O, 0.1 ug/L of vitamin B₁₂, 0.1 ug/L of Biotin, 10 ug/L of thiamine.HCl, pH 7.0)
(2) Culture temperature: culturing can be performed at 15° C. to 25° C.
(3) Culture period: for 2 to 4 weeks
(4) Culture method: stationary culture is suitable.
(5) Light-requiring properties: Necessary. Intensity of light: 4000 lux to 15000 lux. Bright and dark cycle: 12 hours for time for bright period and 12 hours for time for dark period.

As microalgae having the taxonomically same properties as those of the FFG039 strains, microalgae which belong to the genus Chlorococcum sp. and of which the 18S rRNA gene has sequence identity of at least 99.94% with polynucleotide formed of a base sequence of SEQ ID No: 2 are included.

[Floating Culture]

In the present invention, the culturing of microalgae in a medium in a state of being dispersed is referred to as floating culture. In the present invention, the culturing on the liquid surface is not referred to as floating culture. The floating culture is not performed in a primary culture process, but can be used in accordance with the purpose in a pre-culture process.

[Liquid Surface-Floating Culture]

In the present invention, a culture method for culturing microalgae on the liquid surface is referred to as liquid surface-floating culture. Even if microalgae exist on the bottom surface of a culture vessel, on a side surface thereof, on other surfaces, in the middle of a medium, or the like at the same time, the culture method is also referred to as liquid surface-floating culture in a case where the main purpose of the culture method is to perform culturing on the liquid surface. In addition, a large amount of bubble exists on the liquid surface together with a biofilm, and therefore, in some cases, the position of the liquid surface is not always clear. In addition, in some cases, the biofilm is slightly sunk under the liquid surface due to its own weight. Such a case is included as well as a complete liquid surface when the top of the liquid surface is mentioned in the present invention. However, a culture method in which microalgae are cultured in either or only both of the middle of a solution or the bottom surface of a culture vessel is not included in the liquid surface-floating culture.

The liquid surface in the present invention refers to a typical liquid surface of a liquid medium to be described below, and in general, refers to an interface between the liquid medium and the air. In addition, in a case where water is a main component, the liquid surface refers to a water surface.

In addition, when liquid surface-floating culture is performed in the present invention, in some cases, a phenomenon in which a pleat-like structure enters the middle of a liquid from a biofilm on the liquid surface. In the present invention, culturing in such a situation is also included in the liquid surface-floating culture.

Seed algae for performing liquid surface-floating culture may be subjected to suspension treatment, and then, may be added to a culture vessel. Alternately, after adding seed algae to the culture vessel, the seed algae may be stirred in order to accelerate mixing of the seed algae and a liquid medium.

[Pre-Culture Process]

The pre-culture process of the present invention is a process of increasing the number of microalgae until the primary culture can be performed, by causing microalgae for storage, which have been obtained after the completion of a purification process, to proliferate. Any well-known culture method can be selected as the culture method of the pre-culture process. For example, a dispersion culture method or an adhesion culture method, liquid surface-floating culture which has been developed by the present inventors, or a culture method of the present invention can be performed. In addition, the pre-culture process may be performed several times in order for microalgae to proliferate until the microalgae reach a scale in which primary culture can be performed. In addition, in the pre-culture process, stationary culture or non-stationary culture such as shaking culture may be performed in accordance with the purpose. In addition, the pre-culture process may be performed plural times in order for microalgae to proliferate until the microalgae reach a scale in which primary culture can be performed.

In addition, culturing can be performed in either of an indoor place or an outdoor place using a culture vessel having a surface area less than or equal to 1 cm² to 1 m².

[Primary Culture Process]

The primary culture process referred to in the present invention is a culture process which is performed after the pre-culture process is performed and immediately before the final collecting process is performed. The “first culture process”, the “second culture process”, and the third and subsequent processes correspond to the primary culturing process. Depending on the density of microalgae when starting the culturing, the primary culture process can be completed when a sufficient amount of microalgal biofilm is formed on the liquid surface. The primary culture process can be completed in a few days to a few weeks, and more specifically, in 5 days to 4 weeks.

The first culture process refers to a process from the preparation of a suspension liquid and the start of culturing, to the formation of a microalgal biofilm on the liquid surface. The second culture process refers to a process from the start of culturing using microalgae, which remain on the bottom surface or the like without being collected after the first culture process, as seed algae, to the formation of a microalgal biofilm on the liquid surface. Similarly, an n-th culture process refers to a process from the start of culturing using microalgae, which remain on the bottom surface or the like, as seed algae after an (n−1)th culture process, to the formation of a microalgal biofilm on the liquid surface. When second and subsequent culturing is mentioned, the second culture process is included therein, and a third culture process and a fourth culture process are included therein if they exist. In addition, in some cases, the first culture process is called a first primary culturing process in order to clearly distinguish the first culture process from the pre-culturing process. The same principle also applies to the second and subsequent culture processes.

In addition, a culture vessel having a surface area greater than or equal to 100 cm² is generally used. It is possible to perform the culturing in either of an indoor place or an outdoor place, but it is preferable to perform the culturing outdoors.

In addition, the primary culturing process may be repeated many times. In the present invention, it is necessary to perform the primary culture at least twice or more times.

[Seed Algae]

Seed algae of the present invention refer to microalgae which are used when starting the above-described pre-culture process or primary culture process, and become a base for culturing microalgae in the pre-culture process or the primary culture process. Furthermore, in some cases, some microalgae obtained in the collecting process are used as seed algae.

[Bottom Surface Algae]

Bottom surface algae of the present invention indicate microalgae existing in the vicinity of the bottom surface of a culture vessel. The bottom surface algae include algae which are adhered to the bottom surface and are not peeled off from the bottom surface by slight liquid flow, or non-adhesive bottom surface algae which exist in the vicinity of the bottom surface and are moved even by light liquid flow. In addition, the bottom surface algae which have been separated from a microalgal biofilm through a collection operation and have been sunk to the vicinity of the bottom surface can also be included in the non-adhesive bottom surface algae in the present invention.

In the schematic view of the present invention, it is illustrated that the supply of microalgae is performed also from the bottom surface to the top of the liquid surface. However, in reality, there are microalgae in the medium even at locations other than the liquid surface and the bottom surface at a low concentration, and there is a possibility that these microalgae may become a supply source of seed algae. In addition, there are two cases for the supply of microalgae from the bottom surface of the culture vessel to the top of the liquid surface, including a case in which microalgae are moved to the top of the liquid surface without actually being accompanied by proliferation of microalgae on the bottom surface and a case in which the microalgae proliferate while being moved from the bottom surface to the top of the liquid surface.

[Use of Microalgae on Bottom Surface as Seed Algae]

According to an example of the present invention, a suspension liquid or a dispersion liquid which contains microalgae is prepared by dispersing microalgae, which have been obtained through a purification process, in a liquid medium containing an artificial medium; a microalgal biofilm is formed on the liquid surface of the liquid surface by performing culturing in a culture vessel; and a microalgal biofilm is formed on the liquid surface again by culturing microalgae mainly remaining on the bottom surface as seed algae after collecting microalgae proliferating on the liquid surface of the culture vessel.

The greatest characteristics of the present invention is to reuse the microalgae on the bottom surface remaining after the microalgae on the liquid surface are collected, as seed algae. Accordingly, the primary culturing process is performed twice or more times in the present invention. In some cases, initial culturing is called first culturing, and a method, in which algae on the liquid surface which have obtained by performing culturing through the first culturing are collected and algal bodies remaining on the bottom surface are cultured, is called second culturing.

In the present invention, it is possible to reuse a medium. That is, if there are nutritional components remaining in a medium, the culturing may be continued using the used medium as it is, a part of the used medium may be discarded, or a fresh medium may be added thereto. Regarding the amount of the fresh medium added, the same amount of solution as the amount of the medium discarded may be added thereto, or a smaller amount or a larger amount of the fresh medium than the amount of the medium discarded may be added thereto. The addition of a fresh medium is more preferable from the viewpoint that it is possible to improve the proliferation rate of microalgae in the primary culture in a later stage.

In a case of adding a fresh medium thereto, the fresh medium may be added thereto in a state in which nutrient components are dissolved in a liquid, or may be added thereto as a solid content. In a case of adding the fresh medium as a solid content, in some cases, the medium needs to be stirred, and therefore, it is more preferable to add the medium in a state of being dissolved in a liquid.

When adding a medium, a medium having the same components as those in a medium used in primary culture in a previous stage may be used, or a medium having the different components from those in a medium used in primary culture in a previous stage may be added.

In the present invention, it is possible to add a fresh medium, in which the concentration of a specific component is high with respect to a medium which has been used in culturing, after collecting microalgae on the liquid surface. The specify component is not particularly limited, and examples thereof include a compound containing any one selected from the group consisting of nitrogen, phosphorus, potassium, calcium, magnesium, sulfur, manganese, and iron.

In a case of adding a fresh medium, in which the concentration of a specific component is high, to a medium which has been used in culturing, the concentration is preferably 1.01 times to 100000 times that of a usual case, more preferably 10 times to 10000 times that of a usual case, and most preferably 50 times to 5000 times that of a usual case.

If the concentration of a specific component of a fresh medium is 1.01 times less than that of a usual case, a large amount of water is required for preparing the medium. Furthermore, due to various problems, for example, transport properties becoming more deteriorated as the distance between a place at which a fresh medium is prepared and a place in which a culture vessel is installed becomes longer, various devices for supplying a fresh medium to a fresh medium storage container or a culture vessel or the like are required in order to introduce a large amount of medium into the culture vessel. Furthermore, in a case where the amount of medium within a culture vessel is increased, for example, a 1.01-times concentrated solution is added to the culture vessel, the concentration of the medium becomes weak by being approximately halved, in a case where the components of the medium within the culture vessel are almost zero. Accordingly, there is a high possibility that the proliferation rate or the amount of proliferation will be decreased.

If the concentration of a specific component of the medium is 100000 times greater than that of a usual case, the amount of medium solution becomes extremely small and the transport properties or the efficiency of introducing a concentrated medium into a culture vessel are improved. However, solubility is deteriorated depending on the kinds of components of the medium. Furthermore, unnecessary precipitates are generated when introducing the medium into the culture vessel, and in some cases, the precipitates are not dissolved even after the medium is diluted through agitation within the culture vessel. As a result, in some cases, the proliferation rate or the amount of proliferation of microalgae is decreased.

A fresh medium having the same component composition as that of the medium within the culture vessel can be used, but a fresh medium formed of different components from each other may be added. Furthermore, a fresh medium of which the some components are the same as those of the medium within the culture vessel. In addition, it is also possible to use a fresh medium which has been prepared by setting the final concentration after the addition to be significantly different from that of the components of the medium within the culture vessel.

Removal of a medium may be performed from any portion, but it is preferable to remove the medium from any place except for a bottom surface portion, from the viewpoint of suppressing leakage of microalgae in the bottom surface portion. In addition, in a case where there is at least a part of microalgal biofilm on the liquid surface and the microalgae on the liquid surface are also used as seed algae; the quantity of microalgae existing in an intermediate region except for the liquid surface and the bottom surface portion is small, and therefore, it is preferable to collect the microalgae from this region. That is, the removal of a medium can be performed such that at least a part of the medium existing in a region between the liquid surface and the bottom surface is removed, and is preferably performed such that at least a part of the medium existing in a region where microalgae do not substantially exist or microalgae are not visually observed, out of region particularly between the liquid surface and the bottom surface. It is expected that there are zoospores in the region where microalgae do not substantially exist or microalgae are not visually observed. However, the zoospores are quite small compared to algal bodies, and therefore, are not visually observed.

In a case of using microalgae on the bottom surface as seed algae, a part of bottom surface algae may be peeled off to be dispersed in a medium. This is because, by doing this, it is possible to bring microalgae in a state in which only a part of algal body cannot come into contact with the medium, come into contact with a larger amount of the medium, and therefore, it is possible to favorably improve the proliferation rate.

Non-adhesive microalgae existing on the bottom surface may be removed. This is because if microalgae exist unnecessarily on the bottom surface, a decrease in the proliferation rate, which is considered to be caused by unnecessary consumption of nutrient components, can be seen. In addition, the amount of bottom surface algae which exist and are used as seed algae may be adjusted. This is because, by doing this, it is possible to perform appropriate culturing. The quantity of microalgae existing on the bottom surface when starting the culturing is preferably greater than or equal to 0.001 μg/cm², more preferably greater than or equal to 0.1 μg/cm², still more preferably greater than or equal to 0.1 mg/cm², still more preferably greater than or equal to 1 mg/cm², and still more preferably greater than or equal to 2 mg/cm². If the quantity of microalgae thereof is greater than or equal to 0.001 μg/cm², it is possible to increase the proportion of the quantity of microalgae before and after the culturing within a short period of time, which is preferable. In either case, the quantity of microalgae existing on the bottom surface when starting the culturing is preferably less than or equal to 100 mg/cm², more preferably less than or equal to 10 mg/cm², and still more preferably less than or equal to 5 mg/cm².

[Suspension Treatment]

In the present invention, a sample of microalgae subjected to suspension treatment may be used. This is because, with the suspension treatment, the microalgae in a solution are uniformized and the film thickness after culturing is uniformized, and as a result, in some cases, the quantity of microalgae per culturing area increases. Any well-known method can be used for the suspension treatment, and examples thereof include gentle treatment such as treatment of pipetting or shaking a solution of microalgae put into a container by hand and treatment using a stirrer chip or a stirring rod; strong treatment such as ultrasonic treatment or high speed-shaking treatment; and a method of using a substance such as an enzyme decomposing an adhesion substance such as an intracellular matrix.

[Culture Vessel]

Any well-known shape can be used as the shape of a culture vessel (culture pond) as long as the culture vessel can hold a medium. For example, it is possible to use a culture vessel having a cylindrical shape, a rectangular shape, a spherical shape, a plate shape, a tubular shape, and an irregular shape such as plastic bag. In addition, various well-known methods using types such as an open pond type, a raceway type, and a tube type (J. Biotechnol., 92, 113, 2001) can be used. Examples of the forms that can be used as the culture vessel include culture vessels disclosed in Journal of Biotechnology 70 (1999) 313-321, Eng. Life Sci. 9, 165-177 (2009). Among these, use of the open pond type or the raceway type is preferable in view of costs.

As the culture vessel that can be used in the present invention, any of an open type and a closed type can be used. However, the closed-type culture vessel is preferably used in order to prevent diffusion of carbon dioxide to the outside of the culture vessel when the concentration of carbon dioxide, which is higher than that in the air, is used. By using the closed-type culture vessel, it is possible to prevent microorganisms other than microorganisms for culturing, or debris from being mixed in; to suppress evaporation of a medium, and to minimize an adverse effect of wind on a biofilm structure. However, in a case of performing commercial production, culturing in an open system is preferable from the viewpoint that the construction costs are reduced.

[Substrate]

The substrate in the present invention is a solid substance used in FIG. 1E or 1G. As the shape of the substrate, any shape such as a film shape, a plate shape, a fibrous shape, a porous shape, a convex shape, and a wavy shape may be used. In terms of ease of transfer and ease of collecting microalgae from a substrate, a film shape or a plate shape is preferable.

[Material]

The materials of the culture vessel and the substrate that can be used in the present invention are not particularly limited, and well-known materials can be used. For example, it is possible to use a material formed of an organic polymer compound, an inorganic compound, metal, or a composite thereof. In addition, it is also possible to use a mixture thereof

Polyethylene derivatives, polyvinyl chloride derivatives, polyester derivatives, polyamide derivatives, polystyrene derivatives, polypropylene derivatives, polyacrylic derivatives, polyethylene terephthalate derivatives, polybutylene terephthalate derivatives, nylon derivatives, polyethylene naphthalate derivatives, polycarbonate derivatives, polyvinylidene chloride derivatives, polyacrylonitrile derivatives, polyvinyl alcohol derivatives, polyethersulfone derivatives, polyarylate derivatives, allyl diglycol carbonate derivatives, ethylene-vinyl acetate copolymer derivatives, fluorine resin derivatives, polylactic acid derivatives, acrylic resin derivatives, ethylene-vinyl alcohol copolymers, ethylene-methacrylic acid copolymers, and the like can be used as the organic polymer compound.

Glass, ceramics, concrete, and the like can be used as the inorganic compound.

Alloys such as iron, aluminum, copper, or stainless steel can be used as a metallic compound.

Among the above, it is preferable that a part of the material of the substrate or the culture vessel is formed of at least one selected from glass, polyethylene, polypropylene, nylon, polystyrene, vinyl chloride, and polyester.

In addition, the materials of the culture vessel and the substrate may be the same as each other or different from each other.

In addition, in a case of using a closed-type culture vessel, the light receiving surface may be made of a material through which light is transmitted, and a transparent material is more preferable.

[Medium (Liquid Medium)]

In the present invention, any well-known medium (liquid medium) can be used as long as it is possible to culture microalgae. Examples of well-known media include an AF-6 medium, an Allen medium, a BBM medium, a C medium, a CA medium, a CAM medium, a CB medium, a CC medium, a CHU medium, a CSi medium, a CT medium, a CYT medium, a D medium, an ESM medium, an f/2 medium, an HUT medium, an M-11 medium, an MA medium, an MAF-6 medium, an MF medium, an MDM medium, an MG medium, an MGM medium, an MKM medium, an MNK medium, an MW medium, a P35 medium, a URO medium, a VT medium, a VTAC medium, a VTYT medium, a W medium, a WESM medium, an SW medium, and an SOT medium. Among these, freshwater media are an AF-6 medium, an Allen medium, a BBM medium, a C medium, a CA medium, a CAM medium, a CB medium, a CC medium, a CHU medium, a CSi medium, a CT medium, a CYT medium, a D medium, an HUT medium, an M-11 medium, an MA medium, an MAF-6 medium, an MDM medium, an MG medium, an MGM medium, an MW medium, a P35 medium, a URO medium, a VT medium, a VTAC medium, a VTYT medium, a W medium, an SW medium, and an SOT medium. As media for culturing the above-described AVFF007 strains, a C medium, a CSi medium, and a CHU medium, and a mixture of these media are preferable. It is desirable to select the medium in accordance with the types of microalgae to be cultured.

The media may be subjected to ultraviolet ray sterilization, autoclave sterilization, or filter sterilization, or may not be sterilized.

Different media may be used as media in the pre-culture process, the first primary culturing process, and the second primary culture process. In addition, different media may be used during the culture processes.

[Carbon Dioxide]

It is necessary to supply carbon dioxide for culturing a large quantity of microalgae.

In a case of performing dispersion culture in a pre-culture process, carbon dioxide may be supplied to a medium through bubbling which is a conventional method. However, in a case of performing liquid surface-floating culture, it is preferable to supply carbon dioxide from a gas phase. This is because, if carbon dioxide is supplied to a medium through a method such as bubbling, there is a possibility that the structure of a microalgal biofilm on the liquid surface may be destroyed, unevenness in the quantity of algal bodies may occur, the efficiency of collecting a biofilm on a substrate through a collecting process may be deteriorated, and therefore, the quantity of algal bodies collected may decrease.

In the present invention, it is possible to use carbon dioxide in the air, but it is also possible to use carbon dioxide having a higher concentration than that in the air. In this case, it is desirable to perform culturing in a closed-type culture vessel or in a culture vessel which is covered with a coating material such as an agricultural film, in order to prevent the loss of carbon dioxide due to diffusion. The concentration of carbon dioxide in this case is not particularly limited as long as it is possible to achieve the effect of the present invention, but is preferably greater than or equal to the concentration of carbon dioxide in the air and less than 20 volume %, more preferably 0.01 volume % to 15 volume %, and still more preferably 0.1 volume % to 10 volume %. In addition, carbon dioxide may be discharged using a combustion device. In addition, carbon dioxide may also be generated using a reagent.

[Light Source and Amount of Light]

As light sources that can be used in the present invention, any known light source can be used. However, it is possible to use sunlight, LED light, a fluorescent lamp, an incandescent lamp, xenon lamp light, a halogen lamp, and the like. Among these, it is preferable to use sunlight as natural energy, an LED having a good luminous efficiency, or a fluorescent lamp that can be simply used.

The amount of light is preferably 100 lux to 1000000 lux and more preferably 300 lux to 500000 lux. The most preferable amount of light is 1000 lux to 200000 lux. If the amount of light is greater than or equal to 100 lux, it is possible to culture microalgae, and if the amount of light is less than or equal to 1000000 lux, there is a little adverse effect on culturing due to photolesion.

Light may be radiated through any method such as continuous irradiation, and repetition of irradiation and non-irradiation at a constant time interval, but it is preferable that light be turned on and off at a time interval of 12 hours.

The wavelength of light is not limited, and any wavelength can be used as long as the wavelength is a wavelength at which photosynthesis can be performed. A preferred wavelength is a wavelength of sunlight or a wavelength similar to that of sunlight. An example in which the growth rate of photosynthetic organisms is improved by radiating a single wavelength has been reported, and even in the present invention, it is possible to use such an irradiation method.

[Other Culture Conditions]

In the present invention, the pH of a liquid medium (hereinafter, the liquid medium is also referred to as a culture solution) used in a pre-culture process or a primary culture process is preferably within a range of 1 to 13, more preferably within a range of 3 to 11, still more preferably within a range of 5 to 9, and most preferably within a range of 6 to 8.

In addition, it is preferable to select the pH of the medium in accordance with the types of microalgae since a preferred pH is changed in accordance with the types of microalgae. The pH of the liquid medium refers to the pH when starting the culturing. In addition, in some cases, the pH during a culture process is changed accompanying the culturing, and therefore, the pH during the culture process may be changed.

In the present invention, it is possible to add a substance, which has a buffer action, to a medium for maintaining a constant pH in the medium. Accordingly, in some cases, it is possible to suppress a problem in which the pH in a medium is changed in accordance with the progress of culturing of microalgae, or to suppress the phenomenon in which the pH is changed due to supply of carbon dioxide to the medium. As the substance having a buffer action, it is possible to use a well known substance. The use thereof is not limited, but it is possible to suitably use 4-(2-hydroxyethyl)-1-piperazineethanesulfonic acid (HEPES), a sodium phosphate buffer solution, a potassium phosphate buffer solution, or the like. The concentrations or the kinds of buffer substances can be determined in accordance with the types or the culture environments of the microalgae.

If the water depth of a liquid medium is deep in a case where dispersion culture is performed, light does not reach the depth and there is a problem that the stirring efficiency is deteriorated, and therefore, there is restriction. However, in a case of liquid surface-floating culture, microalgae proliferate on the liquid surface at a high density, and therefore, it is unnecessary to supply light to the depth of a culture vessel. Moreover, since it is not essential to perform stirring, it is possible to make the water depth shallow. Accordingly, in terms of the amount of water used becoming small and the handling efficiency becoming good, it is preferable to make the water depth shallow. The water depth is preferably greater than or equal to 0.4 cm, more preferably 1.0 cm to 10 m, still more preferably 2.0 cm to 1 m, and most preferably 4.0 cm to 30 cm. If the water depth is greater than or equal to 0.4 cm, it is possible to form a biofilm, and if the water depth is less than or equal to 10 m, the handling becomes easy. If the water depth is 4.0 cm to 30 cm, an influence due to evaporation of moisture is minimized and the handling of a solution containing a medium or microalgae becomes easy.

The culture temperature can be selected in accordance with the types of microalgae and is not particularly limited. However, the culture temperature is preferably 0° C. to 90° C., more preferably 15° C. to 50° C., and most preferably greater than or equal to 20° C. and less than 40° C. If the culture temperature is greater than or equal to 20° C. and less than 40° C., it is possible to make microalgae suitably proliferate.

If the lower limit input microalgae quantity of microalgae, that is, the quantity of microalgae used when starting culturing is one within a range of culturing, microalgae can proliferate as long as time is given, and therefore, there is no particular restriction. However, the lower limit input microalgae quantity thereof is preferably greater than or equal to 1 cells/cm³, more preferably greater than or equal to 1000 cells/cm³, and still more preferably greater than or equal to 1×10⁴ cells/cm³. Regarding the upper limit input microalgae quantity of microalgae, microalgae can proliferate at any high concentration, and therefore, there is no particular restriction. However, the upper limit input microalgae quantity thereof is preferably lower than or equal to 1×10⁹ cells/cm³, more preferably lower than or equal to 1×10⁸ cells/cm³, and still more preferably lower than or equal to 5×10⁷ cells/cm³ from the viewpoint that, when the concentration thereof is higher than or equal to a certain concentration, the ratio of the quantity of microalgae after the proliferation to the number of input microalgae decreases as the quantity of microalgae becomes higher.

The pre-culture period and the primary culture period in the present invention can be selected in accordance with the types of microalgae and is not particularly limited. However, the pre-culture period and the primary culture period is preferably 1 day to 300 days, more preferably 3 days to 100 days, and still more preferably 7 days to 50 days.

[Size and Proliferation Rate of Microalgal Biofilm Formed on Liquid Surface]

The size of a microalgal biofilm is preferably greater than or equal to 0.1 cm², more preferably greater than or equal to 1 cm², still more preferably greater than or equal to 10 cm², and most preferably the same as the area of the liquid surface of a culture vessel. If the size thereof is greater than or equal to 0.1 cm², it is possible to increase the ratio of the quantity of microalgae when finishing culturing to the quantity of microalgae when starting culturing within a short period of time, which is preferable. In addition, a plurality of microalgal biofilms may exist within a culture region.

The thickness of a microalgal biofilm is preferably within a range of 1 μm to 10000 μm, more preferably within a range of 1 μm to 1000 μm, and most preferably within a range of 10 μm to 1000 μm. If the thickness thereof is within a range of 10 μm to 1000 μm, the strength of the biofilm is high, and therefore, it is possible to harvest a sufficient amount of biofilm.

In a case where the biofilm according to the present invention is a steric three-dimensional structure which is formed such that a part or a plurality of parts of a film-like structure rise in a bubble shape, in general, the height of the three-dimensional structure having a liquid surface of a medium as a reference is preferably within a range of 0.01 mm to 100 mm, more preferably within a range of 0.1 mm to 20 mm, and most preferably within a range of 5 mm to 20 min. If the height thereof is within a range of 5 mm to 20 mm, it is possible to sufficiently decrease the moisture content and to suppress the height of a culture vessel to be low.

In addition, microalgae according to the present invention preferably have a high proliferation rate on the liquid surface. The proliferation rate (that is, an average proliferation rate per day during a period of a logarithmic proliferation phase) of microalgae in the logarithmic proliferation phase is preferably greater than or equal to 0.1 g/m²/day by dry weight, more preferably greater than or equal to 0.5 g/m²/day by dry weight, still more preferably greater than or equal to 1 g/m²/day by dry weight, and most preferably greater than or equal to 3 g/m²/day by dry weight. The proliferation rate of microalgae in the logarithmic proliferation phase is generally less than or equal to 1000 g/m²/day by dry weight.

The weight of dry alga bodies of the biofilm according to the present invention per unit area is preferably greater than or equal to 0.001 mg/cm², more preferably greater than or equal to 0.1 mg/cm², and particularly preferably greater than or equal to 1 mg/cm². The most preferred weight of the dry alga bodies is greater than or equal to 5 mg/cm². This is because it is expected that if the weight of the dry alga bodies per unit area is great, the amount of biomass, such as oil, which has been obtained will become great. In general, the weight of the dry alga bodies of a biofilm per unit area is less than or equal to 100 mg/cm².

Microalgae capable of forming a biofilm, which has the above-described structure, or the area, the thickness, the height, the proliferation rate, and the dry weight of the algal bodies per unit area within the above-described ranges, on the liquid surface, are preferable as the microalgae of the present invention for the same reasons described above.

In addition, only a biofilm on the liquid surface may be collected, or both of the biofilm on the liquid surface and a part of microalgae on the bottom surface may be collected. This is because it is possible to use both of the microalgae on the liquid surface and the microalgae on the bottom surface as biomass. However, in general, when considering oil as a useful substance, the content of oil of the microalgae on the bottom surface becomes lower than that of the microalgae on the liquid surface. Accordingly, the collection of bottom surface algae may be avoided as much as possible.

[Collection]

It is possible to collect a biofilm on the liquid surface in a state in which the liquid surface in the culture vessel is partially covered with the biofilm, but it is preferable to collect the biofilm after the entirety of the liquid surface in the culture vessel is covered with the biofilm in view of obtaining a large quantity of algal bodies of microalgae. In addition, the collection may be performed after continuing culture for a while after the entirety of the liquid surface is covered with the biofilm.

Particularly, it is preferable to perform the collection after a three-dimensional structure is formed on the liquid surface. The three-dimensional structure is a structure which can be seen when a film-like structure further proliferates. In the three-dimensional structure, a larger quantity of microalgae which can be collected and lower moisture content are preferable in comparison to the two-dimensional film-like structure.

As the above-described collection method, greater than or equal to 70% of a biofilm formed on the liquid surface is preferably collected, greater than or equal to 80% thereof is more preferably collected, greater than or equal to 90% thereof is still more preferably collected, and 100% thereof is most preferably collected. The collection rate of a biofilm formed on the liquid surface can be confirmed by, for example, being visually recognized.

[Collection of Microalgal Biofilm on Liquid Surface using Transferring Method]

The transferring method is a process of transferring a biofilm of microalgae on the liquid surface to a first substrate as shown in FIGS. 1D and 1E, and is a type of adhesion which is performed without substantial proliferation.

The first substrate is gently inserted with respect to the liquid surface so as to be parallel to or at an angle close to the liquid surface, and the microalgal biofilm on the liquid surface is adhered to surface of the first substrate. When performing the insertion, the first substrate is slightly obliquely inserted with respect to the liquid surface, and is then finally made to be parallel to the liquid surface. Then, it is possible to collect a large amount of microalgal biofilm with a smaller number of times of transferring, which is preferable. The transferring may be performed plural times in terms of improving the transferring rate.

[Collection of Microalgal Biofilm on Liquid Surface Using Deposition Method]

The method for collecting microalgae on the liquid surface using a second substrate as shown in FIG. 1F is a collection method using a deposition method. The method is a method for obliquely inserting the second substrate with respect to a microalgal biofilm on the liquid surface of a culture vessel, inserting and pulling the second substrate so as to follow the surface of the biofilm, and collecting the biofilm while depositing the biofilm on the surface of the second substrate, as shown in the drawing.

In the drawing, the second substrate is moved from the right side to the left side, but the movement direction of the second substrate may be a reverse direction (that is, movement from the left side to the right side), and the collection may be performed plural times. This is because the collection rate is improved by performing the collection plural times. In a case of performing the collection plural times, the second substrate may be used in a state in which a biofilm is adhered to the second substrate as it is, or a new second substrate may be prepared and used. In addition, although only one sheet of a second substrate is illustrated in FIGS. 1A to 1H, a plurality of sheets of the second substrates may be simultaneously used. Accordingly, the collection rate is improved. Among these, it is preferable to remove a collected biofilm using one sheet of the second substrate, and then, to use the one sheet of the second substrate for the next collection as much as the strength of the second substrate allows, in terms of installation costs of a collection device. In addition, the size of the second substrate, the angle or the movement rate of the second substrate with respect to the liquid surface, or the like can be freely set in accordance with the purpose. FIG. 1G is in a state in which a biofilm is collected on the second substrate.

The size of the second substrate can be appropriately changed in accordance with the size of a culture vessel. However, it is preferable to use a second substrate having a size slightly smaller than that of the minor diameter of the inner wall of the culture vessel. This is because, by doing this, it is possible to avoid unnecessary contact of the second substrate with the inner wall of the culture vessel when moving the second substrate, and an escape of a microalgal biofilm on the liquid surface during collection via a gap between the culture vessel and the second substrate is hardly generated.

In addition, in some cases, a microalgal biofilm proliferating on the liquid surface within a culture vessel grows from a film shape to a pleat shape depending on the culturing state. In this case, it is possible to collect the pleat-like biofilm by making the insertion dept of the second substrate into a liquid deep.

[Detachment of Microalgal Biofilm from Substrate]

Detachment is a part of a collection process. Any method may be used as the method for detaching a microalgal biofilm from a substrate as long as the method used is a method in which it is possible to peel off microalgae on a substrate. It is possible to peel off a biofilm by applying a stream of water; subjecting a container, into which a substrate is put, to ultrasonic treatment; vigorously shaking the container, into which a substrate is put, after closing a lid of the container; performing high speed-shaking treatment; or using a cell scraper. Among these, a method for peeling off a biofilm from a substrate using a holding device, in which a material which does not damage a substrate is used, for example, a cell scraper, is preferable.

Furthermore, it is also possible to peel off a microalgal biofilm from the top of a substrate by simply inclining the substrate. This method is the most preferred method due to its simplicity.

In addition, the substrate may be re-used many times.

[Dry Alga Bodies]

The dry alga bodies in the present invention are obtained by drying a collected substance of microalgae which has been obtained by the present invention.

Any well-known method can be used as the method for drying the collected substance of microalgae as long as it is possible to reduce the moisture in the collected substance of microalgae, and there is no particular restriction. Examples thereof include a method of sun-drying a Collected substance of microalgae; a method of heat-drying a collected substance of microalgae; a method of freeze-drying a collected substance of microalgae; and a method of blowing dry air onto a collected substance of microalgae. Among these, the freeze-drying method is preferable in view of being capable of suppressing decomposition of components contained in a collected substance of microalgae, and the heat-drying method or the sun-drying method is preferable in view of being capable of efficiently perform the drying in a short period of time.

[Moisture Content]

The moisture content of the present invention is obtained by dividing the weight of moisture contained in a collected substance by the weight of the collected substance, and then multiplying the result by 100. The moisture content of a microalgal biofilm in the present invention is preferably 99% to 60%, more preferably 95% to 80%, and most preferably 90% to 85%.

The moisture content in a case where microalgae are collected using a centrifugal separator after being cultured through dispersion culture is generally set to about 90%, and the moisture content of a biofilm on the liquid surface obtained through a culture method in the present invention is lower than the value, which is excellent compared to a conventional method. The moisture content of a three-dimensional structure is lower than that of a film-like structure. It is estimated that this is because the three-dimensional structure is away from the liquid surface and is close to a light source, and therefore, drying is progressed to some extent.

[Useful Substance]

The useful substance in the present invention is one type of biomass derived from microalgae and is the name of a substance beneficial to an industry which is obtained through a process such as an extraction process from biomass and a purification process. Such a substance includes a final product, an intermediate, or a raw material of a pharmaceutical product, cosmetics, or a health food, or the like; a raw material, an intermediate, or a final product of a chemical synthetic substance; a hydrocarbon compound; an energy-alternative substance such as oil, alcohol compound, hydrogen, or methane; enzyme; protein; nucleic acid; a lipid compound such as sugar or DHA; and astaxanthin. The useful substance can be accumulated in microalgae through a useful substance accumulation process.

[Biomass and Oil]

The biomass in the present invention refers to a renewable organic resource derived from organisms excluding a fossil resource, and examples thereof include substances, food products, materials, fuel, and resources derived from organisms. As the algal biomass, residues of microalgae after the microalgae itself (which may also have a biofilm shape) and the useful substance have been collected are included.

Oil in the present invention refers to a flammable fluid substance, is a compound mainly formed of carbon and hydrogen, and is a substance occasionally containing oxygen atom, nitrogen atom, and the like. In general, oil is a mixed substance and is a substance which is extracted using a low-polarity solvent such as hexane or acetone. There is a case in which the composition thereof is forming of hydrocarbon compounds, fatty acids, triglycerides, or the like, and a case in which oil is formed of a plurality of types of compositions selected therefrom. In addition, oil can be esterified to be used as biodiesel.

The method of collecting a useful substance and oil contained in a collected substance of microalgae is not particularly limited as long as the method does not impair the effect of the present invention.

As a general method for collecting oil, dry alga bodies are obtained by heat-drying a final collected substance, and then, oil is extracted using an organic solvent after performing cell-disruption. In general, the extracted oil contains impurities such as chlorophyll, and therefore, purification is performed. There is a case of performing purification through silica gel column chromatography or performing purification through distillation (for example, a distillation method disclosed in JP2010-539300A). In the present invention, it is possible to use such a method as well.

In addition, there is also a method of extracting oil in algal bodies using an organic solvent after crushing microalgae through an ultrasonic treatment or dissolving microalgae using protease or an enzyme (for example, a method disclosed in JP2010-530741A). In the present invention, it is possible to use such a method as well.

In addition, it is preferable that the biofilm according to the present invention has high oil content in view of usefulness as biomass. Specifically, the oil content per dry alga body of the biofilm is preferably higher than or equal to 5 mass %, more preferably higher than or equal to 10 mass %, and particularly preferably higher than or equal to 15 mass %. In general, the oil content per dry alga body of the biofilm is lower than or equal to 80 mass %.

### Example

The present invention will be further described in detail with reference to the following Examples, but the present invention is not limited to the following Examples.

### Example 1

Stationary culture was performed as pre-culture at a concentration of carbon dioxide of 5% at 23° C. under irradiation with a fluorescent lamp (light irradiation by turning on and off the fluorescent lamp every 12 hours) at 15000 lux after putting a mixture of 40 mL of CSiFF04 medium (FIG. 2) and FFG039 strains (at a concentration of algal bodies of 0.032 mg/mL) into polystyrene case no. 28 (4-5605-05, As One Corporation) which was then put into a vacuum desiccator (1-070-01, As One Corporation). Light on the side surface and the bottom surface of the polystyrene case no. 28 was blocked with black plastic cases. The state immediately before the culturing was shown in FIG. 8.

14 days after the culturing, a culture vessel was taken out of the vacuum desiccator, and a microalgal biofilm on the surface of water was collected using a nylon film having the same length as a short side of the polystyrene case no. 28.

The biofilm was put into a 5 mL tube for homogenizing together with a small amount of CSiFF04 medium, the tube for homogenizing (TM-655, Tomy Seiko Co., Ltd.) was set in a beads-type cell disrupter MS-100 (Tomy Seiko Co., Ltd.), and homogenization treatment lasting for 20 seconds was performed three times at 4200 rpm to obtain a suspension liquid a of microalgae. However, beads were not used.

This solution was diluted and the turbidity was calculated by measuring the absorbance at 660 nm. 740 mL of a suspension liquid b at a concentration of 0.032 mg/mL was obtained by calculating the quantity of algal bodies of the above-described suspension liquid a from a relational expression between the turbidity and the number of algal bodies which have been calculated in advance, and by diluting the suspension liquid a with the CSiFF04 medium.

Liquid surface-floating culture was performed as first primary culture through the same method as that in the pre-culture after putting 40 mL of the suspension liquid b into a polystyrene case no. 28.

7 days after the culturing, the whole microalgal biofilm on the surface of water was collected through the same method as that in the pre-culture. This test was set as a test 1A.

The weight of the collected substance was measured and the weight of the collected substance after freeze-drying was further measured. The dry weight and the moisture content were calculated from these measurement values. The collected amount (dry weight per unit area) in the test 1A became 0.239 mg/cm².

After removing the medium within the culture vessel, algal bodies existing on the bottom surface were collected and the quantity of algal bodies was calculated based on the weight of the collected substance after freeze-drying, and became 0.132 mg/cm². That is, 0.132 mg/cm² corresponds to the quantity of algal bodies input in tests 1B, 1C, 1D, and 1E.

Three samples out of samples which were obtained by collecting microalgae on the surface of water, were put into a vacuum desiccator, and second primary culture was performed under the same culture conditions as those in the pre-culture. However, the culture period was set to 14 days. This test was set as a test 1B. That is, the test is Example in a case of performing no replacement of a medium and no treatment on bottom surface algae. The collected amount became 3.324 mg/cm².

Bottom surface algae in three containers out of samples, which were obtained by collecting microalgae on the surface of water, were peeled off from the bottom surface of each culture vessel using a cell scraper, and were then put into a vacuum desiccator, and second primary culture was performed under the same culture conditions as those in the pre-culture. However, the culture period was set to 14 days. This test was set as a test 1C. That is, the test is Example in a case of performing no replacement of a medium, but performing dispersion treatment on the bottom surface algae. The collected amount became 3.382 mg/cm².

Media in three containers out of samples, which were obtained by collecting microalgae on the surface of water, were removed as much as possible, and 40 mL of a fresh CSiFF04 medium was added to each of the containers such that the structure of bottom surface algae is not destroyed. Thereafter, the containers were put into a vacuum desiccator and second primary culture was performed under the same culture conditions as those in pre-culture. However, the culture period was set to 14 days. This test was set as a test 1D. That is, the test is Example in a case of performing replacement of a medium, but performing no dispersion treatment on the bottom surface algae. The collected amount became 4.964 mg/cm². The collected amount when being combined with the collected amount in the first culture became 5.203 mg/cm².

Media in three containers out of samples, which were obtained by collecting microalgae on the surface of water, were removed as much as possible, and 40 mL of a fresh CSiFF04 medium was added to each of the containers. Thereafter, the bottom surface algae were peeled off from the bottom surface of each culture vessel using a cell scraper, and were then put into a vacuum desiccator, and the second primary culture was performed under the same culture conditions as those in pre-culture. However, the culture period was set to 14 days. This test was set as a test 1E. That is, the test is Example in a case of performing replacement of a medium and performing dispersion treatment on the bottom surface algae. The collected amount became 5.603 mg/cm². The collected amount when being combined with the collected amount in the first culture became 5.842 mg/cm².

The test, in which second primary culture was continuously performed for 14 days without collecting microalgae 7 days after the first primary culture, was set as a test 1F. The collected amount became 4.607 mg/cm².

In this Example, microalgae on the liquid surface were collected through a deposition method unless otherwise stated.

From the above, it was found that it was possible to perform culturing using bottom surface algae as seed algae and that the quantity of microalgae on the surface of water was increased by performing replacement of a medium in a case of starting culturing using bottom surface algae again, and by performing dispersion treatment.

### Example 2

### In Case of Repeatedly Performing Primary Culture

Pre-culture was performed through the same method as that in Example 1. However, AVFF007 strains were used as microalgae.

170 mL of a suspension liquid b of microalgae at 5×10⁵ cells/mL was obtained through the same method as that in Example 1.

Liquid surface-floating culture was performed as first primary culture through the same method as that in the pre-culture after putting 40 mL of the suspension liquid b into a polystyrene case no. 28.

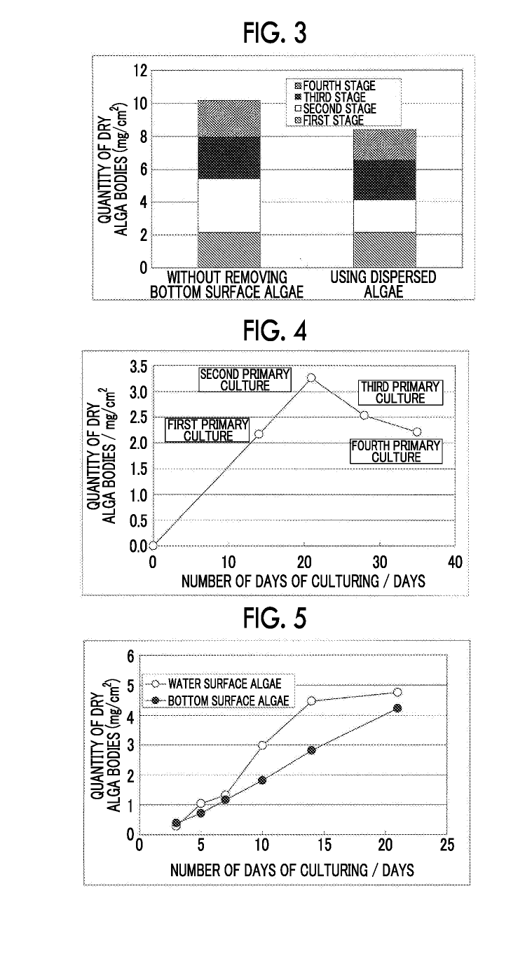

14 days after the culturing, microalgae on the surface of water were collected through the same method as that in Example 1. As shown in FIG. 3, the quantity of algal bodies (collected amount) was 2.16 mg/cm².

The following two kinds of samples were prepared for second primary culture.

First, after collecting microalgae on the surface of water, the medium was removed as much as possible. At this time, microalgae on the bottom surface were made to remain within a container. In addition, a splinter-like microalgal biofilm, which was thought to be less adhered to the bottom surface, was also made to remain within the container as much as possible. Next, 40 mL of a fresh CSiFF04 medium was put into the container so as not to peel off the microalgal structure on the bottom surface. That is, culturing was started by having the microalgae on the bottom surface as seed algae.

The other sample was prepared through the same method′ as that in the first primary culture. That is, 90 mL of a suspension liquid of microalgae at the same concentration as that of the suspension liquid b of microalgae was newly prepared. 40 mL of the new suspension liquid was put into each new polystyrene case no. 28.

Culturing was performed on the culture vessel, which has been prepared as above, under the culture conditions through the same method as that in Example 1.

All of water surface algae derived from the samples were collected 21 days (the number of days of culturing after the start of the first primary culture) after the culturing. The collected amount of the former sample was 3.26 mg/cm² and the collected amount of the latter sample was 1.96 mg/cm². The culture period of the latter sample was 7 days.

In third primary culture, samples were prepared through the same method as that in the second primary culture. 28 days after the culturing, all of microalgae on the surface of water were collected. As a result, the collected amount of the former sample was 2.54 mg/cm² and the collected amount of the latter sample was 2.4 mg/cm². The culture period of the latter sample was 7 days.

In fourth primary culture, samples were prepared through the same method as that in the second primary culture. 35 days after the culturing, all of microalgae on the surface of water were collected. As a result, the collected amount of the former sample was 2.22 mg/cm² and the collected amount of the latter sample was 1.88 mg/cm². The culture period of the latter sample was 7 days.

From the above, in a case where microalgae on the bottom surface were used as seed algae, the total quantity of dry alga bodies of all of water surface algae became 10.18 mg/cm². In contrast, in a case where culturing was newly started by preparing a suspension liquid of microalgae, the total quantity of dry alga bodies thereof became 8.4 mg/cm². Accordingly, it was found that starting culturing from bottom surface algae is preferable since the amount of biomass obtained is large as well as it is unnecessary to prepare seed algae.

The quantity of dry alga bodies (collected amount) with respect to the number of days of culturing in the case of using bottom surface algae as seed algae were plotted in FIG. 4. The quantity of dry alga bodies was increased up to the second culturing, but the quantity of dry alga bodies obtained was decreased from the third culturing.

### Example 3

### Relationship Between Algae on Surface of Water and Quantity of Microalgae on Bottom Surface

Pre-culture was performed through the same method as that in Example 2 to obtain 1000 mL of a suspension liquid b of algal bodies. 40 mL of the suspension liquid b of algal bodies was put into each 24 polystyrene cases no. 28, and liquid surface-floating culture was performed through the same method as that in Example 2. Each of three culture vessels was taken out of a vacuum desiccator after the number of days of culturing on day 3, day 5, day 7, day 10, day 14, and day 21. Microalgae on each surface of water and microalgae on each bottom surface were collected and the quantity of dry alga bodies thereof was measured. The results were shown in FIG. 5. The quantity of algal bodies of microalgae on the surface of water was increased in accordance with the number of days of culturing. In addition, samples having various quantities of algal bodies were obtained through this test.

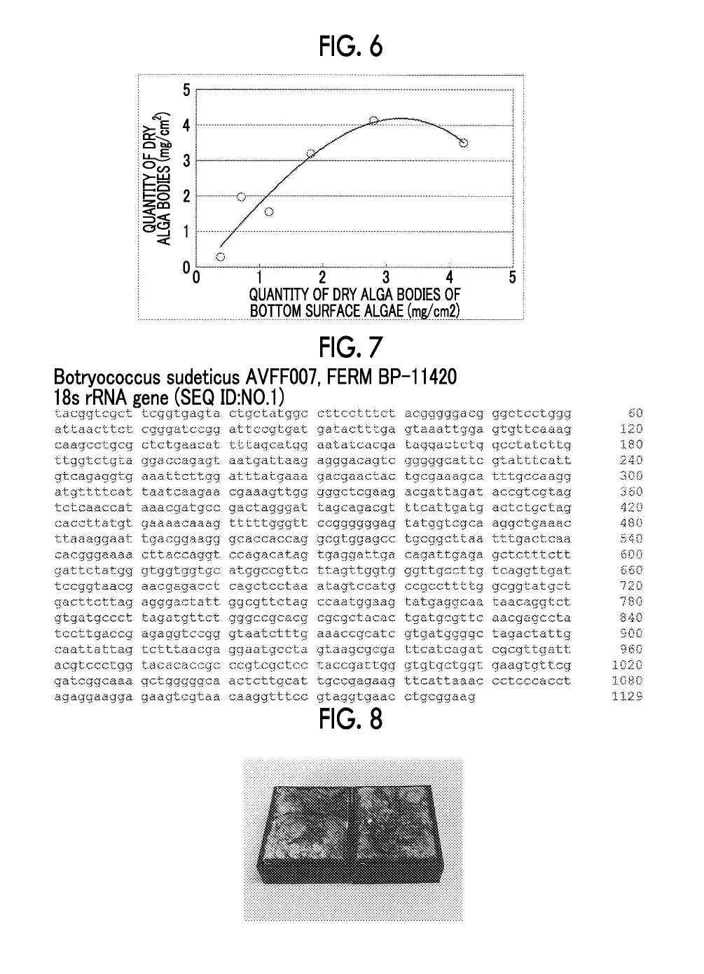

Culturing was started after removing a medium in a state in which microalgae on the bottom surface remain as much as possible in the same manner as in Example 2 and putting 40 mL of a fresh CSiFF04 medium thereinto. The culturing was stopped 7 days after culturing, that is, 10 days to 28 days after culturing in the total culture period, and the quantity of dry alga bodies of microalgae on the surface of water was measured. The results were shown in FIG. 6. 3 mg/cm² was the most favorable in the quantity (that is, quantity of seed algae) of microalgae on the bottom surface.

Accordingly, it became clear that there is an optimum value in the quantity of microalgae on the bottom surface and a larger quantity of algal bodies is not always favorable.

### Example 4

### Suitability of Removal of Non-Adhesive Microalgae

Pre-culture and first primary culture were performed through the same method as that in Example 2.

The following two kinds of samples were prepared after collecting microalgae on the surface of water.

A first sample was a sample into which a fresh CSiFF04 medium was put after removing free microalgae which were not adhered on the bottom surface when removing a medium, and a second sample was a sample into which a fresh CSiFF04 medium was put after making as much microalgae, which were adhered and not adhered on the bottom surface, as possible remain within a culture vessel when removing a medium.

The quantity of dry alga bodies in the former case was 0.453 mg/cm² and the quantity of dry alga bodies in the latter case was 1.658 mg/cm².

Second primary culture was performed through the same method as that in Example 2. Microalgae on the surface of water were collected 11 days after the culturing (in total, 25 days after the culturing from the first primary culture). As a result, the quantity of dry alga bodies in the former case became 4.0 mg/cm² and the quantity of dry alga bodies in the latter case became 3.25 mg/cm².

From the above, it was found that the quantity of algal bodies of microalgae after the culturing was increased in the sample from which free microalgae, that is, non-adhesive bottom surface algae were removed. In the case of FIG. 2 in Example 2, non-adhesive microalgae on the bottom surface were not removed, and therefore, the quantity of microalgae on the surface of water was decreased in accordance with the increase of the number of times of culturing. It is considered that such a result is shown because nutrient components in the medium are more quickly consumed as the quantity of seed algae becomes larger.

### Example 5

### Method for Adjusting Quantity of Seed Algae by Removing Microalgae On Bottom Surface

Pre-culture and first primary culture were performed through the same method as that in Example 2. However, FFG039 strains were used as microalgae. It was confirmed that there was an escape of 0.5 mg of dry alga bodies with respect to one container during collection performed through a transferring method.

The following three kinds of samples were prepared after collecting microalgae on the surface of water.

In a first sample, after collecting microalgae on the surface of water, microalgae on the bottom surface were peeled off along the long side of a culture vessel, using a cell scraper (MS-93100, manufactured by Sumitomo Bakelite Co., Ltd.) with a width of 10 mm. That is, microalgae on the bottom surface were peeled off over the width of 1 cm out of the width of 4.6 cm of the culture vessel. Next, as much medium as possible was removed together with the peeled off microalgae. Next, 40 mL of a fresh CSiFF04 medium was put into the culture vessel. Accordingly, about 22% of bottom surface algae can be removed.

In a second sample, microalgae on the bottom surface were removed through the same method as that in the first sample, but the removal of bottom surface algae was performed twice using a cell scraper. That is, about 43% of bottom surface algae were removed.

In a third sample, only replacement of a medium was performed without performing the removal of bottom surface algae.

Second culture was performed through the same method as that in Example 2 using the samples prepared in this manner.

The quantity of dry alga bodies of collected substances in each of the first, second, and third samples became 7.54 mg/cm², 6.92 mg/cm², and 6.43 mg/cm². From the above, it was found that it was possible to improve the quantity of microalgae on the surface of water through controlling the quantity of algal bodies of microalgae on the bottom surface, and that it was possible to use a holding device such as a cell scraper for the method for controlling the quantity of algal bodies thereof.

### Example 6

### Method for Leaving Bottom Surface Algae in Method for Replacing Medium in Region Between Bottom Surface and Liquid Surface

First primary culture was performed through the same method as that in Example 2. However, FFG039 strains were used as the species of algae.

Culturing was performed as second primary culture through the same method as that in the first primary culture by collecting microalgal biofilm on the surface of water and replacing a medium with a fresh CSiFF04 medium. However, the replacement of the medium was performed on day 7 of the culture period. That is, the CSiFF04 medium was replaced with a medium, from which Ca(NO₃)₂.4H₂O and KNO₃ were removed, using a region which was thought to have a small quantity of microalgae existing in areas between where water surface algae and bottom surface algae exist. The amount of solution of the replaced medium was about 30 mL (75% at the time of starting culturing). In contrast, a sample on which no replacement of a medium was performed was also prepared.

After performing the culturing for 14 days in total as second primary culture, the microalgae on the surface of water were collected and the collected substance was freeze-dried. Then, the quantity of dry alga bodies and the oil content were measured.

The quantity of dry alga bodies of the sample on which replacement of a medium was performed became 7.86 mg/cm² and the quantity of dry alga bodies of the sample on which no replacement of a medium was performed became 8.01 mg/cm². The oil content of each of the dry alga bodies was 39.2% and 28.4%. In addition, the moisture contents thereof were respectively 82% and 85%.

From the above, it became clear that it was possible to perform replacement of a medium using the region between water surface algae and bottom surface algae.

### Example 7

### In Case of Adding High Concentration Medium Component

First primary culture was performed under the same conditions as those in Example 1. A culture vessel was prepared by pasting an agricultural film on an upper edge of a plastic rectangular container (upper portion (95 cm×65.8 cm×29.3 cm), lower portion (92.5 cm×59.1 cm)) using double-sided tape, and the first primary culture was performed using 27.5 L of a culture solution (water depth of 5 cm). A vacuum desiccator was not used since a coating film was used. Moreover, a shading tool was not used since the container itself has light shielding properties on the side surface and the bottom surface. The culturing was performed for 21 days. In addition, homogenization treatment was not performed. In addition, culturing was performed using five culture vessels 6A to 6E at the same time.

21 days after the culturing, microalgae on the liquid surface of the culture vessels 6A to 6E were collected and the collected algal bodies were dried. As a result, an average of the dry weight of the collected algal bodies was 22.2 g. The whole medium of the culture vessel 6E was removed and microalgae remaining on the bottom surface of the culture vessel were collected. Then, the dry weight thereof was measured, and as a result, was 0.896 g. Accordingly, in the culture vessels 6A to 6D, there are seed algae, at almost the same quantity as that in the culture vessel 6E, which are required for next culture.

Components of a medium at a higher concentration than that of a CSiFF04 medium were added to the culture vessel 6A from which the microalgae on the liquid surface were collected. Specifically, 45 mL of Ca(NO₃)₂.4H₂O (45 ml of an aqueous solution at a concentration of 750×610.5=457.8 mg/L was added to about 27 L of a culture system) at 610.5 times concentration, 90 mL of KNO₃ at 305 times concentration, and 25 mL of other components at 1100 times concentration were added thereto. The reason that the concentration magnification varies is that the degree of solubility (dissolution time) of each reagent varies with respect to water.

Components of a medium at a higher concentration than that of the CSiFF04 medium were added to the culture vessel 6B from which the microalgae on the liquid surface were collected. Specifically, 45 mL of Ca(NO₃)₂.4H₂O at 610.5 times concentration, 180 mL of KNO₃ at 152 times concentration, and 25 mL of other components at 1100 times concentration were added thereto.

Components of a medium at a higher concentration than that of the CSiFF04 medium were added to the culture vessel 6C from which the microalgae on the liquid surface were collected. Specifically, 45 mL of Ca(NO₃)₂.4H₂O at 610.5 times concentration, 360 mL of KNO₃ at 76 times concentration, and 25 mL of other components at 1100 times concentration were added thereto.

Components of a medium at a higher concentration than that of the CSiFF04 medium were added to the culture vessel 6D from which the microalgae on the liquid surface were collected. Specifically, 45 mL of Ca(NO₃)₂.4H₂O at 610.5 times concentration, 90 mL of KNO₃ at 152 times concentration, 25 mL of MgSO₄.7H₂O at 1100 times concentration, 25 mL of K₂HPO₄ at 1100 times concentration, 25 mL of KH₂PO₄ at 1100 times concentration, and 25 mL of FeCl₃.6H₂O at 1100 times concentration were added thereto. Other components which had been added to the CSiFF04 medium were not added thereto.

Microalgae within the culture vessels 6A to 6D were collected since a biofilm was thinly formed on the liquid surface for 3 days to 5 days after performing culturing, and a thick biofilm was formed on the liquid surface 21 days after the start of the culturing. Each of the collected amounts was 25.9 g, 27.9 g, 23.3 g, and 22.7 g in order, from the culture vessel 6A. Accordingly, the concentration magnification being 152 times was the best with respect to KNO₃.

Similarly, components of a medium at a high concentration were added to the culture vessel 6A. Furthermore, culturing was performed three times. As a result, the weight of microalgae on the liquid surface was 24.3 g, 22.6 g, and 21.2 g.

### Sequence Table Free Text

SEQ ID No: 1: part of base sequence of 18S rRNA gene of AVFF007 strains

SEQ ID No: 2: part of base sequence of 18S rRNA gene of FFG039 strains

What is claimed is:

1. A method for culturing microalgae, comprising:
a first culture step of culturing microalgae in a medium within a culture vessel, forming a biofilm on the liquid surface of the medium, and maintaining the microalgae on the bottom surface of the culture vessel;
a step of collecting at least a part of the biofilm on the liquid surface formed in the first culture step and leaving at least some microalgae on the bottom surface inside the culture vessel; and
a second culture step of culturing the microalgae remaining on the bottom surface within the identical culture vessel, and forming a biofilm on the liquid surface of the medium.

2. The culture method according to claim 1, further comprising:
a step of removing at least a part of the medium used in the first culture step and adding a fresh medium.

3. The culture method according to claim 2,
wherein the concentration of at least one component contained in the fresh medium is at least 50 times the concentration of the medium used in the first culture step.

4. The culture method according to claim 3,
wherein the at least one component which is contained in the fresh medium and of which the concentration is at least 50 times the concentration of the medium used in the first culture step is a compound including any one selected from the group consisting of nitrogen, phosphorus, potassium, calcium, magnesium, sulfur, and iron.

5. The culture method according to claim 2,
wherein the removal of the at least a part of the medium used in the first culture step is removal of at least a part of a medium existing in a region between the liquid surface and the bottom surface.

6. The culture method according to claim 1,
wherein the microalgae on the bottom surface include adhesive microalgae, and
wherein the culture method further includes a step of separating at least some adhesive microalgae from the top of the bottom surface.

7. The culture method according to claim 1
wherein the second culture step includes a step of collecting at least a part of the biofilm formed on the liquid surface and leaving at least some microalgae on the bottom surface inside the culture vessel, and
wherein the culture method further includes third and subsequent culture steps of culturing the remaining microalgae on the bottom surface within the identical culture vessel and repeating formation of a biofilm on the liquid surface of the medium.

8. The culture method according to claim 7,
wherein, in second and subsequent culture steps, the quantity of microalgae existing on the bottom surface as seed algae is less than or equal to 5 mg/cm².

9. The culture method according to claim 8,
wherein, in the second and subsequent culture steps, at least a part of a medium used in an immediately prior culture step is removed, and at this time, the quantity of microalgae existing on the bottom surface as seed algae is set to less than or equal to 5 mg/cm² by removing both non-adhesive microalgae on the bottom surface and the at least a part of the medium thereof.

10. The culture method according to claim 5,
wherein the microalgae on the bottom surface include adhesive microalgae and non-adhesive microalgae, and
wherein, when the at least a part of the medium used in the first culture step are removed, both the non-adhesive microalgae on the bottom surface and the at least a part of the medium thereof are removed.

11. The culture method according to claim 1,
wherein the quantity of microalgae existing on the bottom surface as seed algae is greater than or equal to 0.1 mg/cm².

12. The culture method according to claim 1,
wherein the quantity of microalgae existing on the bottom surface as seed algae is greater than or equal to 2 mg/cm².

13. The culture method according to claim 1,
wherein the microalgae are microorganisms containing oil.

14. The culture method according to claim 1,
wherein the microalgae are green algae.

15. The culture method according to claim 1,
wherein the microalgae belongs to Botryococcus sp., Chlamydomonas sp., Chlorococcum sp., Chlamydomonad sp., Tetracystis sp., Characium sp., or Protosiphon sp.

16. The culture method according to claim 1,
wherein the microalgae belongs to Botryococcus sudeticus or Chlorococcum sp.

17. The culture method according to claim 1,
wherein the microalgae are Botryococcus sudeticus FERM BP-11420 or microalgae having taxonomically the same properties as those of Botryococcus sudeticus FERM BP-11420, or are Chlorococcum sp. FERM BP-22262 or microalgae having taxonomically the same properties as those of Chlorococcum sp. FERM BP-22262.

18. A method for producing algal biomass, comprising:
a culture step including the culture method according to claim 1; and
a step of collecting a biofilm on the liquid surface formed in the second culture step.

19. The production method according to claim 18,
wherein the algal biomass is oil.
